(19) United States
(12) Patent Application Publication
(10) Pub. No.: US 2026/0245733 A1
(43) Pub. Date: Aug. 20, 2026

(54) PREDICTING SUBJECTIVE RECOVERY FROM ACUTE EVENTS USING CONSUMER WEARABLES

(71) Applicant: EVIDATION HEALTH, INC., San Mateo, CA (US)

(72) Inventors: Ieuan CLAY, Munich (DE); Luca FOSCHINI, Santa Barbara, CA (US); Ernesto RAMIREZ, Los Angeles, CA (US); Marta KARAS, Boston, MA (US); Nikki MARINSEK, Albuquerque, NM (US)

(21) Appl. No.: 19/330,065

(22) Filed: Sep. 16, 2025

Related U.S. Application Data
(63) Continuation of application No. 17/946,975, filed on Sep. 16, 2022, now abandoned.
(60) Provisional application No. 63/245,464, filed on Sep. 17, 2021.

(51) Int. Cl. G16H 50/30; G16H 10/60; G16H 50/70

(52) U.S. Cl. CPC G16H 50/30; G16H 10/60; G16H 50/70

(57) ABSTRACT

In an aspect, a method for predicting, for a subject, a recovery time from an acute or debilitating event is disclosed. The method may comprise (i) retrieving wearable sensor data from a first time period and a second time period. The first time period may be prior to the acute or debilitating event. The second time period may be after the acute or debilitating event. The method also may comprise (ii) determining the recovery time for the acute or debilitating event at least in part by processing said wearable sensor data from the first time period and the second time period with a trained machine learning algorithm.

### CROSS-REFERENCE

This application is a continuation of U.S. patent application Ser. No. 17/946,975, filed Sep. 16, 2022, which application claims priority to U.S. Provisional Patent Application No. 63/245,464, filed on Sep. 17, 2021, each of which are incorporated herein by reference in their entirety.

### BACKGROUND

A major challenge in monitoring recovery from acute or debilitating events (e.g., acute illness, surgery, or falls) is the lack of long-term individual baseline data which would enable accurate and objective assessment of functional recovery. Consumer-grade wearable devices, which may enable collection of person-generated health data (PGHD) on virtually all aspects of individual lifestyles and behaviors, may be able to provide this data.

But engagement with healthcare systems, and therefore monitoring, typically only begins when an individual is diagnosed or their symptoms otherwise become so severe that they seek care. An advantage of PGHD captured via consumer-grade wearables is that prediction or forecasting of outcomes can leverage data collected prior to the diagnosis or event, enabling early detection and treatment by “funneling” high risk individuals towards proactive screening.

Assessment of recovery may be highly challenging, primarily because canonical practice may provide no personalized baseline to which functional recovery can be compared. Equally, subjective (i.e., patient-reported) assessment of recovery may be challenging due to individual reference perceptions and expectations of what “normal” (i.e., fully recovered function) is. While evidence exists that increasing activity during rehabilitation improves recovery outcomes, triggering these interventions may be practically difficult. For example, functional recovery from lower-limb surgeries may take six months, e.g., for knee and hip replacement or hip fracture surgery. For such conditions, recovery trajectories longer than six months are typically seen as abnormal and a trigger for further intervention.

### SUMMARY

Recovery for acute health conditions may be assessed relative to a personal baseline derived from long-term passive monitoring with consumer wearables. Person-generated health data (PGHD) from consumer-grade technologies can capture, and be used to predict, long-term recovery trajectories. This work may help to identify patients at risk for delayed rehabilitation early enough to trigger additional or more targeted rehabilitation interventions. Personalized recommendations based on individualized baseline data can be a major contribution of PGHD towards virtual healthcare.

There is a need for a system that can use person-generated health data from consumer-grade technologies (e.g., wearable devices) to predict a time to recovery from a debilitating event. The debilitating event may be a health condition or health intervention. The health condition may be an illness or injury. The intervention may be a surgery.

In an aspect, a method for predicting, for a subject, a recovery time from an acute or debilitating event is disclosed. The method comprises (i) retrieving wearable sensor data from a first time period and a second time period. The first time period is prior to the acute or debilitating event and wherein the second time period is after the acute or debilitating event. The method also comprises (ii) determining the recovery time for the acute or debilitating event at least in part by processing said wearable sensor data from the first time period and the second time period with a trained machine learning algorithm.

In some embodiments, the wearable sensor data comprises health measurements.

In some embodiments, the health measurements comprise at least one of sleep efficiency, step count, and heart rate.

In some embodiments, the health measurements comprise at least two of sleep efficiency, step count, and heart rate.

In some embodiments, the sensor data is collected daily throughout the first time period and the second time period.

In some embodiments, the first time period is longer than, the same length, or shorter than the second time period.

In some embodiments, the machine learning algorithm is an ensemble learning method.

In some embodiments, the machine learning algorithm uses one or more decision trees.

In some embodiments, the machine learning algorithm is random forests.

In some embodiments, the machine learning algorithm uses boosted trees.

In some embodiments, the machine learning algorithm uses gradient boosted trees.

In some embodiments, the machine learning algorithm is XGBoost.

In some embodiments, the method further comprises generating a recovery score from the wearable sensor data. Generating the recovery score comprises (i) generating a similarity group of a plurality of subjects sharing at least one characteristic with the subject, wherein the at least one characteristic relates to health data, personal data, or demographic data. Generating the recovery score also comprises (ii) calculating a ranking for the subject with respect to the similarity group. The ranking relates to (1) a type of wearable sensor data or (ii) a weighted combination of types of wearable sensor data. Generating the recovery score also comprises (iii). calculating the recovery score at least in part from the ranking.

In some embodiments, the method further comprises providing the ranking or the score to a graphical user interface (GUI).

In some embodiments, the trained machine learning algorithm is produced by: (i) maintaining, for each of a plurality of human subjects, (1) a self-reported time to recovery and (2) wearable sensor data from a first period and a second period; and (ii) training the machine learning algorithm to predict the self-reported time to recovery from the wearable sensor data.

In an aspect, a system for predicting a time to recovery from an acute or debilitating event for a subject is disclosed. The system comprises (i) a wearable device comprising one or more sensors, the one or more sensors configured to collect health data from the subject, wherein the health data is collected during a first time period and a second time period. The system also comprises (ii) a server comprising one or more processors for processing the health data from the first time period and the second time period using a machine learning algorithm. The processing produces a predicted time to recovery. The system also comprises (iii) a client device for providing the predicted time to recovery to the subject via a graphical user interface (GUI).

In some embodiments, the wearable device is a smart watch.

In some embodiments, the one or more sensors comprises at least one of a heart rate sensor, a step count sensor, or a sleep sensor.

In some embodiments, the one or more sensors comprises at least two of a heart rate sensor, a step count sensor, or a sleep sensor.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory comprises machine executable code that, upon execution by the one or more computer processors, implements any of the methods above or elsewhere herein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 illustrates a filtering process for an experiment to predict times to recover of human subjects;

FIG. 2 illustrates changes in activity features baseline to representative features from step, heart rate, and sleep data for periods before and after surgery;

FIG. 3 illustrates plots that show average trajectories of daily number of steps across three self-reported recovery time groups, across three lower limb surgeries;

FIG. 4 illustrates an explainable process determining features important for driving predictive power of a machine learning model;

FIGS. 5A-5F illustrate examples of a user interface from an application for reporting medical care;

FIG. 6 illustrates a plot of step data density for a plurality of patients;

FIG. 7A illustrates a four-piecewise fit used in a change point (CP) detection procedure;

FIG. 7B illustrates an example trajectory of a likelihood of a main change point;

FIG. 8 illustrates a plot showing a set of likelihood trajectories;

FIG. 9 illustrates a chart of assumed wearable PGHD availability in a set of predictive modeling experiment scenarios;

FIG. 10 illustrates a plot showing a change in a daily total number of steps pre-surgery and post-surgery;

FIG. 11A illustrates a plot showing estimated trajectories of a daily number of steps across two self-reported recovery time groups pre-surgery and post-surgery;

FIG. 11B illustrates a plot showing estimated trajectories of a daily number of steps across four self-reported recovery time groups pre-surgery and post-surgery;

FIG. 12 illustrates a system for predicting a time to recovery for a subject;

FIG. 13 illustrates a process for predicting post-procedure recovery time;

FIG. 14 illustrates screen captures of a user interface providing a score to a subject; and

FIG. 15 shows a computer system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

### Overview

The disclosed method uses machine learning to better understand how particular patients may respond to surgical or medical procedures, or acute debilitating events. Using data collected by wearable sensors from patients with different physical characteristics and personal attributes, the disclosed method may predict a patient's time to recovery. The system may use a machine learning model trained on patient wearable device sensor data collected prior to and following an event. Based at least in part on analysis of this wearable device sensor data, which may include, but is not limited to, step count, heart rate, and sleep efficiency, the system may make a prediction as to at which point a patient will be fully recovered (i.e., a recovery time or time to recovery).

### Data Collection and Sensors

A wearable device may comprise one or more sensors to measure physical attributes of a human subject. For example, the wearable device may include one or more accelerometers, heart rate sensors, barometers, orientation sensors, or gyroscopes. The wearable device may include one or more cameras (e.g., red-green-blue (RGB), YUV, or depth), radar sensors, microphones, infrared sensors, or sensors configured to measure electromagnetic signals (e.g., electrodes or magnetometers). The sensors may be implantable, physically coupled to the body, or not contacted with the body.

The sensors of the wearable device may be configured to measure one or more quantities indicative of a subject's physical health or biophysical characteristics. For example, the sensors may be configured to measure step count, heart rate, sleep efficiency (the total number of minutes slept divided by the overall time in the bed), sleep quality, disordered sleep, respiration, blood oxygen, blood pressure, pulse rate, body temperature, gaze direction, glucose, or another health-related quantity. In some embodiments, the system may analyze data from at least one of sleep efficiency, step count, and heart rate data. In some embodiments, the system may analyze data from at least two of sleep efficiency, step count, and heart rate data.

The sensors may collect subject health data from before and after a debilitating event. The debilitating event may be a health intervention. The health intervention may be surgery. The surgery may be any surgery that causes a major and short-term disruption in mobility, sleep, or physiology. The surgery may be lower limb surgery. The surgery may be weight loss surgery. In some embodiments, the methods and systems disclosed herein may be configured to predict times to recovery from debilitating events that do not comprise weight loss surgery. The lower limb surgery may be bone repair surgery, ligament surgery, tendon surgery, knee or knee replacement surgery, or hip replacement surgery. The surgery may be open heart surgery, spine or neurosurgery, surgery involving lungs or otherwise the respiratory apparatus.

The recovery time may be from an illness, such as COVID, flu, or another acute condition for which the onset date is known with accuracy. The recovery time may be from a trauma, the trauma may be an injury, the injury may be an ankle sprain, Achilles rupture, or other ligament tear.

Health data may be collected for a first time period before the acute or debilitating event (or “event”) occurs. The health data may be collected at least one week, at least two weeks, at least three weeks, at least four weeks, at least five weeks, at least six weeks, at least seven weeks, at least eight weeks, at least nine weeks, at least ten weeks, at least 15 weeks, at least 20 weeks, at least 25 weeks, or at least 30 weeks before the health procedure. The health data may be collected at most one week, at most two weeks, at most three weeks, at most four weeks, at most five weeks, at most six weeks, at most seven weeks, at most eight weeks, at most nine weeks, at most ten weeks, at most 15 weeks, at most 20 weeks, at most 25 weeks, or at most 30 weeks before the acute or debilitating event. The health data may be collected between one and two weeks, between two and three weeks, between three and five weeks, between five and ten weeks, between ten and fifteen weeks, between 15 and 20 weeks, or between 20 and 30 weeks before the acute or debilitating event.

Health data may be collected for a second time period after the acute or debilitating event (or “event”) occurs. The health data may be collected at least one week, at least two weeks, at least three weeks, at least four weeks, at least five weeks, at least six weeks, at least seven weeks, at least eight weeks, at least nine weeks, at least ten weeks, at least 15 weeks, at least 20 weeks, at least 25 weeks, or at least 30 weeks after the acute or debilitating event. The health data may be collected at most one week, at most two weeks, at most three weeks, at most four weeks, at most five weeks, at most six weeks, at most seven weeks, at most eight weeks, at most nine weeks, at most ten weeks, at most 15 weeks, at most 20 weeks, at most 25 weeks, or at most 30 weeks after the acute or debilitating event. The health data may be collected between one and two weeks, between two and three weeks, between three and five weeks, between five and ten weeks, between ten and fifteen weeks, between 15 and 20 weeks, or between 20 and 30 weeks after the event.

The health data may be collected at a high frequency. For example, the health data may be collected at least once every minute, at least once every ten minutes, at least once every 15 minutes, at least once every 30 minutes, at least once every hour, at least once every two hours, at least once every three hours, at least once every six hours, at least once every 12 hours, at least once every day, or at least once every week. The health data may be collected at most once every six hours, at most once every 12 hours, at most once every day, or at most once every week.

### Other Data

The disclosed machine learning system may use non-wearable data in addition to wearable sensor data when making predictions. For example, the system may use demographic or personal data about a human subject. The data may include age, weight, height, fitness level or exercise frequency, types of exercise performed, gender, sex, location, medical history, family medical history, medications taken, wearable device usage patterns, occupation, or other data.

### Subject

The subject may be a human subject. The subject may be an animal subject. The subject may be a mammalian subject, such as a monkey, ape, mouse, rat, rabbit, dog, cat, pig, sheep, or cow. The subject may be a bird, such as a chicken, duck, or pigeon. The subject may be a reptile, such as a snake, lizard, or crocodilian. The methods disclosed herein may apply to debilitating events faced by animals, such as avian influenza.

In some embodiments, data from the subject, such as time to recovery, may be reported by the subject. In some embodiments, such data may be reported by a health care provider or another third party. In some embodiments, such data may be reported by an automated system.

### Machine Learning Algorithms

The disclosed system may use one or more machine learning algorithms to predict recovery time from sensor data. For example, the disclosed system may use a support vector machine, a logistic regression (e.g., using LASSO), a decision tree method (e.g., gradient boosted trees or random forest), or a neural network (e.g., a recurrent neural network). The system may use deep learning (e.g., a deep neural network).

### System and Method

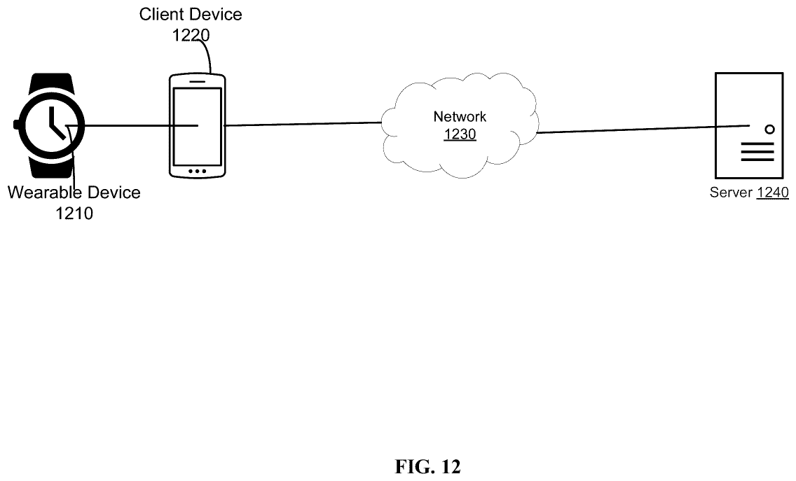

FIG. 12 illustrates a system 1200 for predicting a time to recovery (or recovery time) for a subject. The system may include one or more wearable devices 1210, a client device 1220, a network 1230, and a server 1240.

The wearable device 1210 and the client device 1220 may be coextensive or may be separate devices. In general, the wearable device 1210 may comprise one or more wearable device sensors (also referred to herein as “sensors”) for collecting patient health data and may include a capability to connect to a network (e.g., the network 1230) to transfer the sensor data to other components of the system 1200. The wearable device 1210 may be a watch, headgear, jewelry, clothing, fabric, footwear, headband, eyewear, or other article or electronic device configured to contact the skin of or a body part of the subject, and which may include or may be communicatively coupled to electronic circuitry that may collect, transmit, and/or process electrical signals derived from the subject. For example, the wearable device 1210 may be a Fitbit® or APPLE® Watch. The wearable device may comprise a sleep sensor to measure sleep efficiency, a heart rate sensor to measure heart rate, and/or a step count sensor (e.g., a pedometer) to measure step count.

The client device 1220 may be a computing device configured to access an application enabling a subject to self-report data. The client device 1220 may be a mobile computing device. The client device may be a smartphone, wearable device, cell phone, personal digital assistant (PDA), tablet computer, laptop computer, desktop computer, or other computing device. The application may be installed natively on the client device or may be accessible via a browsing application. The application may enable a subject to self-report recovery from surgery. The application may also enable a subject to track a progression or recovery trajectory from an acute or debilitating event (e.g., a surgery).

The server 1240 may maintain user or subject data and perform analysis of the data. For example, the server may store one or more machine learning models used to perform analysis of wearable data received from the subject as well as, optionally, subject-reported demographic or personal data. The server 1240 may use the machine learning models to make one or more predictions about a time to recovery for one or more users. The server 1240 may be a physical or cloud server. A physical server may comprise one or more computing devices.

The network 1230 may be a hardware and software system configured to enable the computing components of the system 1200 to communicate electronically and share resources with one another. The network 1230 may be the Internet, a local area network (LAN), or a wide area network (WAN).

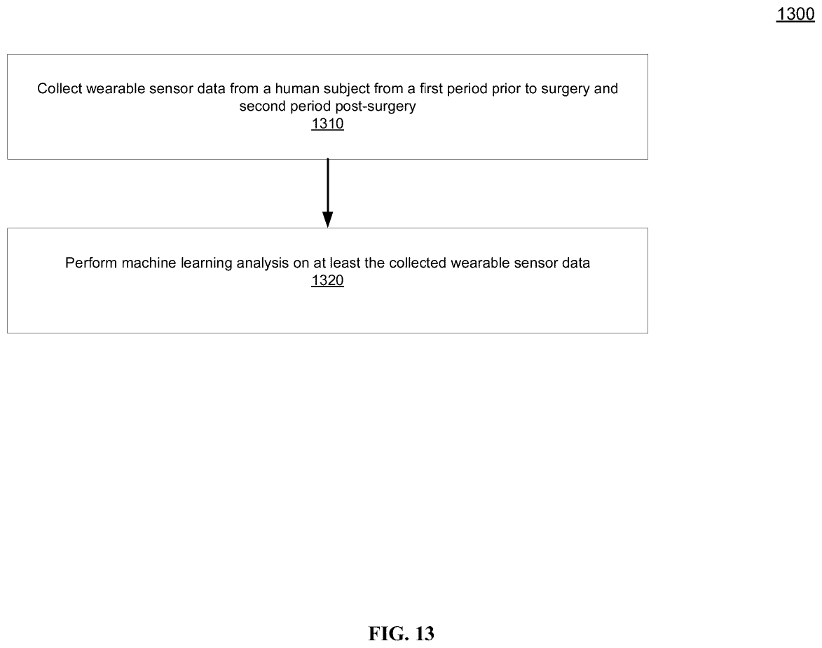

FIG. 13 illustrates a process 1300 for predicting recovery time (or “time to recovery”) from a debilitating or acute event.

In a first operation 1310, the system may collect wearable sensor data from a human subject for a first period prior to an acute or debilitating event and for a second period after the acute or debilitating event. In some embodiments, the first period is shorter than the second period. In some embodiments, the first period is the same length as the second period. In some embodiments, the first period is longer than the second period. The first period may be, for example, 12 weeks prior to surgery. The second period may be, for example, 26 weeks following surgery. The wearable sensor data may be collected daily. The wearable sensor data may comprise subject health measurements. For example, the wearable sensor data may comprise heart rate, step count, and sleep efficiency.

In a second operation 1320, the system may perform machine learning analysis on at least the collected wearable sensor data. The machine learning analysis may comprise a decision tree-based model (e.g., XGBoost). The machine learning analysis may generate a prediction for a post-event recovery time.

The prediction may be a binary prediction. For example, the system may predict a fast recovery time or a slow recovery time. A fast recovery time may be, for example, two months or less. A slow recovery time may be, for example, three months or more.

The prediction may be a multiclass prediction. For example, the system may predict a recovery time which may fall into one of the following categories: zero to one month, one to two months, two to three months, three to four months, or more than four months.

### Recovery Score

In some embodiments, the system may compute a personalized, real-time recovery score for a subject during the recovery period.

For a large database of recovery person-generated health data (PGHD) for a population, the system may, for a particular subject or individual, select a group of 20 similar individuals (“similarity group”) from the population. The similarity of the group may be based on individual characteristics, such as age, gender, type of acute or debilitating event suffered, time elapsed since diagnosis, another statistic, or a combination thereof. The similarity may be assessed using a distance function such as Euclidean, Mahalanobis, cosine similarity, or another function, between the vector representing the characteristic of the individual and the vector representing the same characteristics for other individuals whose similarity is being evaluated. For a particular health statistic or quantity of interest (e.g., step count, heart rate, or sleep efficiency), the system may compute a distribution for the similarity group and rank the subject within the group. For example, within the group, the system may calculate a percentile ranking for step count. The system may also average these rankings to produce an overall score in real-time. The system may also use the probability of full recovery within (e.g., six months) as computed by the machine learning system and may calculate a percentile ranking of that probability within the group. The score may be updated as the system receives additional data (e.g., self-reported or generated by wearable device) from the user.

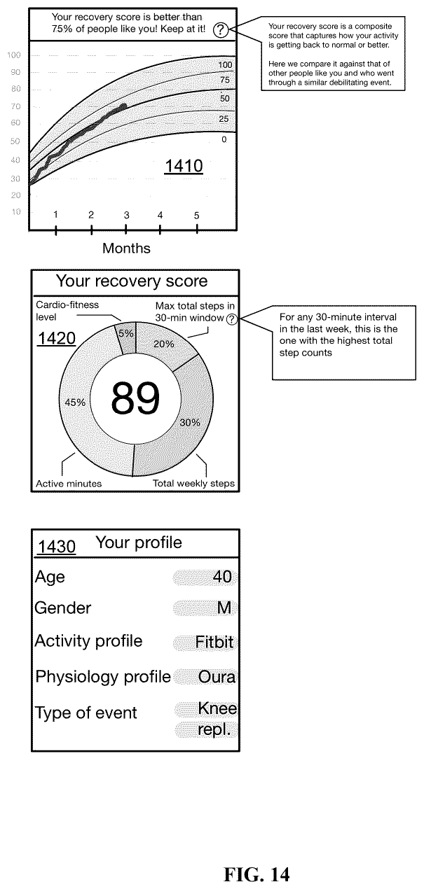

FIG. 14 illustrates screen captures 1410, 1420, 1430 of a user interface providing a score to a subject. The user interface may belong to a mobile device application. In a first screen capture 1410, a user's percentile score over time is overlaid on scores from users in the subject's similarity group. In this particular case, a score may representing probability of full recovery within six months rescaled over the range 0-100, as predicted by the machine learning system based on wearables and self-reported data available at the day the score is computed. The interface may inform the subject that recovery has progressed better than recovery for 75% of users in the similarity group, meaning that their probability of recovery at six months is higher than those of 75% of individuals in a similarity group. In a second screen capture 1420, the user interface displays the components of the subject's recovery score. This score may represent the probability of recovery at six months based on data available at the time the score is produced (e.g., at month three, as represented in the figure). This value may be generated as a prediction by the machine learning system. In this example, the contributions are 5% from cardio-fitness level, 20% maximum steps in a 30-minute window, 30% total weekly steps, and 45% active minutes.

### Machine Learning

a. Training Phase

A machine learning software module may be provided by a server (e.g., the server 1240) and may implement one or more machine learning algorithms. A machine learning software module as described herein is configured to undergo at least one training phase wherein the machine learning software module is trained to carry out one or more tasks including data extraction, data analysis, and generation of output.

In some embodiments of the software application described herein, the software application comprises a training module that trains the machine learning software module. The training module is configured to provide training data to the machine learning software module, the training data comprising, for example, wearable sensor data, the date (e.g., precise to the day), of occurrence of an acute or debilitating event, and ground truth data comprising self-reported times to recovery (or recovery times), once recovery is completed or can no longer be attained (no recovery). In additional embodiments, said training data is comprised of wearable sensor data and recovery times with corresponding subject personal and/or demographic data. In some embodiments of a machine learning software module described herein, a machine learning software module utilizes automatic statistical analysis of data to determine which features to extract and/or analyze from wearable sensor data. In some of these embodiments, the machine learning software module determines which features to extract and/or analyze from subject health data based on the training that the machine learning software module receives.

In some embodiments, a machine learning software module is trained using a data set and a target in a manner that might be described as supervised learning. In these embodiments, the data set is conventionally divided into a training set, a test set, and, in some cases, a validation set. In some embodiments, the data set is divided into a training set and a validation set. A target is specified that contains the correct classification of each input value in the data set. For example, a set of wearable sensor data from one or more individuals is repeatedly presented to the machine learning software module, and for each sample presented during training, the output generated by the machine learning software module is compared with the desired target. The difference between the target and the set of input samples is calculated, and the machine learning software module is modified to cause the output to more closely approximate the desired target value. In some embodiments, a backpropagation algorithm is utilized to cause the output to more closely approximate the desired target value. After many training iterations, the machine learning software module output will closely match the desired target for each sample in the input training set. Subsequently, when new input data, not used during training, is presented to the machine learning software module, it may generate an output classification value indicating which of the categories the new sample is most likely to fall into. The machine learning software module is said to be able to “generalize” from its training to new, previously unseen input samples. This feature of a machine learning software module allows it to be used to classify almost any input data which has a mathematically formulatable relationship to the category to which it should be assigned.

In some embodiments of the machine learning software module described herein, the machine learning software module utilizes an individual learning model. An individual learning model is based on the machine learning software module having trained on data from a single individual and thus, a machine learning software module that utilizes an individual learning model is configured to be used on a single individual on whose data it trained, or on individuals deemed similar to the individual on whose data it trained. Similarity may be defined in terms of a distance function (e.g., Euclidean, Mahalanobis, cosine similarity) between vectors containing variables characterizing two individuals, such as demographics, social determinant of health. It may be defined as distance in the space where those vectors are embedded (e.g., using autoencoder embedding techniques).

In some embodiments of the machine training software module described herein, the machine training software module utilizes a global training model. A global training model is based on the machine training software module having trained on data from multiple individuals and thus, a machine training software module that utilizes a global training model is configured to be used on multiple patients/individuals.

In some embodiments of the machine training software module described herein, the machine training software module utilizes a simulated training model. A simulated training model is based on the machine training software module having trained on data from wearable sensor data. A machine training software module that utilizes a simulated training model is configured to be used on multiple patients/individuals.

In some embodiments, the use of training models changes as the availability of wearable sensor data changes. For instance, a simulated training model may be used if there are insufficient quantities of appropriate patient data available for training the machine training software module to a desired accuracy. As additional data becomes available, the training model can change to a global or individual model. In some embodiments, a mixture of training models may be used to train the machine training software module. For example, a simulated and global training model may be used, utilizing a mixture of multiple patients' data and simulated data to meet training data requirements.

Unsupervised learning is used, in some embodiments, to train a machine training software module to use input data such as, for example, wearable sensor data and output, for example, a predicted recovery time. Unsupervised learning, in some embodiments, includes feature extraction which is performed by the machine learning software module on the input data. Extracted features may be used for visualization, for classification, for subsequent supervised training, and more generally for representing the input for subsequent storage or analysis. In some cases, each training case may consist of a plurality of wearable sensor data.

Machine learning software modules that are commonly used for unsupervised training include k-means clustering, mixtures of multinomial distributions, affinity propagation, discrete factor analysis, hidden Markov models, Boltzmann machines, restricted Boltzmann machines, autoencoders, convolutional autoencoders, recurrent neural network autoencoders, and long short-term memory autoencoders. While there are many unsupervised learning models, they all have in common that, for training, they require a training set consisting of biological sequences, without associated labels.

A machine learning software module may include a training phase and a prediction phase. The training phase is typically provided with data to train the machine learning algorithm. Non-limiting examples of types of data inputted into a machine learning software module for the purposes of training include medical image data, clinical data (e.g., from a health record), encoded data, encoded features, or metrics derived from wearable sensor data. Data that is inputted into the machine learning software module is used, in some embodiments, to construct a hypothesis function to determine a predicted recovery time. In some embodiments, a machine learning software module is configured to determine if the outcome of the hypothesis function was achieved and based on that analysis make a determination with respect to the data upon which the hypothesis function was constructed. That is, the outcome tends to either reinforce the hypothesis function with respect to the data upon which the hypothesis function was constructed or contradict the hypothesis function with respect to the data upon which the hypothesis function was constructed. In these embodiments, depending on how close the outcome tends to be to an outcome determined by the hypothesis function, the machine learning algorithm will either adopt, adjust, or abandon the hypothesis function with respect to the data upon which the hypothesis function was constructed. As such, the machine learning algorithm described herein dynamically learns through the training phase what characteristics of an input (e.g., data) are most predictive in determining whether the features of a patient's wearable data are associated with a particular time to recovery.

For example, a machine learning software module is provided with data on which to train so that it, for example, can determine the most salient features of a received wearable sensor data to operate on. The machine learning software modules described herein train as to how to analyze the wearable sensor data, rather than analyzing the wearable sensor data using pre-defined instructions. As such, the machine learning software modules described herein dynamically learn through training what characteristics of an input signal are most predictive in determining whether the features of wearable sensor data predict a particular time to recovery.

In some embodiments, training begins when the machine learning software module is given wearable sensor data and asked to determine a recovery time. The predicted time to recovery is then compared to the true time to recovery that corresponds to the wearable sensor data. An optimization technique such as gradient descent and backpropagation is used to update the weights in each layer of the machine learning software module to produce closer agreement between the time to recovery predicted by the machine learning software module, and the actual time to recovery. This process is repeated with new wearable sensor data and time to recovery data until the accuracy of the network has reached the desired level. An optimization technique is used to update the weights in each layer of the machine learning software module to produce closer agreement between the time to recovery predicted by the machine learning software module, and the true time to recovery. This process is repeated with new wearable sensor data and time to recovery data until the accuracy of the network has reached the desired level.

In some embodiments, an individual's time to recovery is inputted by the individual of the system (e.g., using a mobile device application). In some embodiments, an individual's time to recovery is inputted by an entity other than the individual. In some embodiments, the entity can be a healthcare provider, healthcare professional, family member or acquaintance. In additional embodiments, the entity can be the instantly described system, device or an additional system that analyzes wearable sensor data and provides data related to time to recovery.

In some embodiments, a strategy for the collection of training data is provided to ensure that the wearable sensor data represents a wide range of conditions to provide a broad training data set for the machine learning software module. For example, a prescribed number of measurements during a set period may be required as a section of a training data set. Additionally, these measurements can be prescribed as having a set amount of time between measurements. In some embodiments, wearable sensor data measurements taken with variations in a subject's physical state may be included in the training data set.

In general, a machine learning algorithm is trained using wearable sensor data and/or any features or metrics computed from the above said data with the corresponding ground-truth values. The training phase constructs a transformation function for predicting a time to recovery from wearable sensor data and/or any features or metrics computed from the above said data of the unknown patient. The machine learning algorithm dynamically learns through training what characteristics of input data are most predictive in determining a time to recovery. A prediction phase uses the constructed and optimized transformation function from the training phase to predict the time to recovery by using the wearable sensor data and/or any features or metrics computed from the above said data of the unknown patient.

b. Prediction Phase

Following training, the machine learning algorithm is used to determine, for example, the time to recovery on which the system was trained using the prediction phase. With appropriate training data, the system can identify the time in the future at which a patient may be expected to recover.

The prediction phase uses the constructed and optimized hypothesis function from the training phase to predict a time to recovery from the wearable sensor data.

In some embodiments, a probability threshold can be used in conjunction with a final probability to determine whether or not the patient is expected to recover within a particular fixed time (e.g., six months). In some embodiments, the probability threshold is used to tune the sensitivity of the trained network. For example, the probability threshold can be 1%, 2%, 5%, 10%, 15%, 20%, 25%, 30%, 35%, 40%, 45%, 50%, 55%, 60%, 65%, 70%, 75%, 80%, 85%, 90%, 95%, 98% or 99%. In some embodiments, the probability threshold is adjusted if the accuracy, sensitivity or specificity falls below a predefined adjustment threshold. In some embodiments, the adjustment threshold is used to determine the parameters of the training period. For example, if the accuracy of the probability threshold falls below the adjustment threshold, the system can extend the training period and/or require additional wearable sensor data and/or times to recovery. In some embodiments, additional measurements and/or times to recovery can be included into the training data. In some embodiments, additional measurements and/or times to recovery can be used to refine the training data set.

### Gradient Boosting

Embodiments of this disclosure may be implemented using gradient boosting algorithms such as XGBoost, a version of the gradient boosting algorithm designed for efficacy, computational speed, and model performance.

Boosting may refer to a technique (e.g., an ensemble learning technique) for increasing performance (e.g., of a machine learning algorithm or model). In some embodiments, boosting may convert a weak hypothesis or weak learner (a learner may be a program used to learn a machine learning model from data) to a strong learner, increasing predictive accuracy of a machine learning model.

Boosting is an ensemble learning method. Ensemble learning is a process in which decisions from multiple machine learning (ML) models are combined to reduce errors and improve prediction when compared to a single ML model. Ensemble learning may use ensemble voting on aggregated decisions from multiple weak learners (which may use decision tree algorithms) to generate a strong prediction. A weak learner may be defined as a program that does not make accurate predictions or produces outputs that have weak correlations with actual or ground truth values. For XGboost or other gradient boosting algorithms, decision trees may form the bases for weak learners. A boosting algorithm may use sequential ensemble learning—i.e., it may create new weak learners and may sequentially combine their predictions to improve model performance. For a sequence of predictors, the boosting algorithm may fit a predictor to residual errors made by the previous predictor.

Predictors in a boosting algorithm may comprise decision trees. A decision tree may be a supervised machine learning algorithm used for predictive modeling of a dependent variable (target) based on input of several independent variables. Decision trees may be classification trees or regression trees. A classification tree may be a decision tree that identifies a class or category in which a fixed or categorical target variable would most likely fall. A regression tree may predict a value of a continuous variable.

Gradient boosting, in particular, is boosting that uses gradient descent to minimize errors. Gradient boosting may adjust weights during training iteratively using a gradient descent algorithm. This method may iteratively reduce the loss of a machine learning model. In this context, loss may be defined as a quantification of a negative consequence associated with a prediction error.

Gradient boosting algorithms may be regression algorithms or classification algorithms. A regression algorithm may use a mean-squared error (MSE) loss function, while a classification algorithm may use a logarithmic loss function.

Gradient boosting uses additive modeling, a process that adds a new decision tree at a time to a gradient boosting model to reduce the loss and therefore improve the predictive power of the model. The additive modeling process may combine the output of each new tree with the combined output of the preceding trees until the model loss is minimized below a threshold or a limit on the number of trees the model can use is reached. Each subsequent predictor that is added may be fit to the residual errors (i.e., the difference between the predicted value and the observed value) made by the previous predictor (assuming a MSE loss function).

Extreme gradient boosting (XGBoost) may enhance gradient boosting with advanced regularization (L1 and L2).

In some embodiments, the machine learning methods disclosed herein are implemented using other ensemble methods, other decision tree methods, or other boosting methods.

### Experiment

The following sections describe setup and results of an experiment and should not be construed to limit this disclosure. Many of the procedures described with respect to this experiment may be used to determine predictions for other debilitating or acute events in addition to lower limb surgery events.

### Methods

Fitbit device data of steps, heart rate and sleep from 26 weeks before to 26 weeks after a self-reported surgery date was collected for 1,324 individuals who underwent surgery on a lower limb. Subgroups of individuals who self-reported surgeries for bone fracture repair (355 individuals), tendon or ligament repair/reconstruction (773), and knee or hip joint replacement (196) were identified. Linear mixed models were used to estimate average effect of time relative to surgery on daily activity measurements while adjusting for gender, age, and participant-specific activity baseline. For example, self-reported recovery time was predicted using XGBoost for a sub-cohort of 127 individuals with dense wearable data who underwent tendon or ligament surgery.

### Results

The 1,324 study individuals were all U.S. residents, predominantly female (84%), white or Caucasian (85%) and young to middle-aged (mean 36.2 years). In some embodiments, 12-week pre- and 26-week post-surgery trajectories of daily behavioral measurements (step count, heart rate, sleep efficiency score) captured activity changes relative to an individual's baseline. Recovery trajectories differ across surgery types, recapitulate the documented effect of age on functional recovery, and highlight differences in relative activity change across self-reported recovery time groups. Finally, in the case of a sub-cohort of 127 individuals, long-term recovery can be accurately predicted, on an individual level, only 1 month after surgery (AUROC 0.734, AUPRC 0.8). In some embodiments predictions are most accurate when long-term, individual baseline data are available.

### Data Collection

The experiment used an online platform where people can connect their digital health tools, including wearable activity trackers and fitness apps. This platform enables rapid recruitment of participants to specific studies, where consent for all research is granted on a per use basis.

Data was collected from a previously cited study, surveying participant experience relating to surgery and medical devices. Briefly, participants were asked about which surgeries they had experienced, and for the most recent surgery, the type of surgery, the date of surgery and the time required for recovery. The full survey is included in Supplementary Note 1. Between May 5 and September 21 of 2018, 200,325 individuals consented to take part in the study. 50,938 participants reported they underwent a medical procedure, out of which 4,312 reported at least one of the three lower limb procedures itemized in the survey (surgery to repair a bone fracture, tendon or ligament repair/reconstruction surgery, or knee or hip joint replacement surgery). The initial dataset consisted of 3,740 participants reporting lower limb surgery as their most recent surgery.

### Data Processing

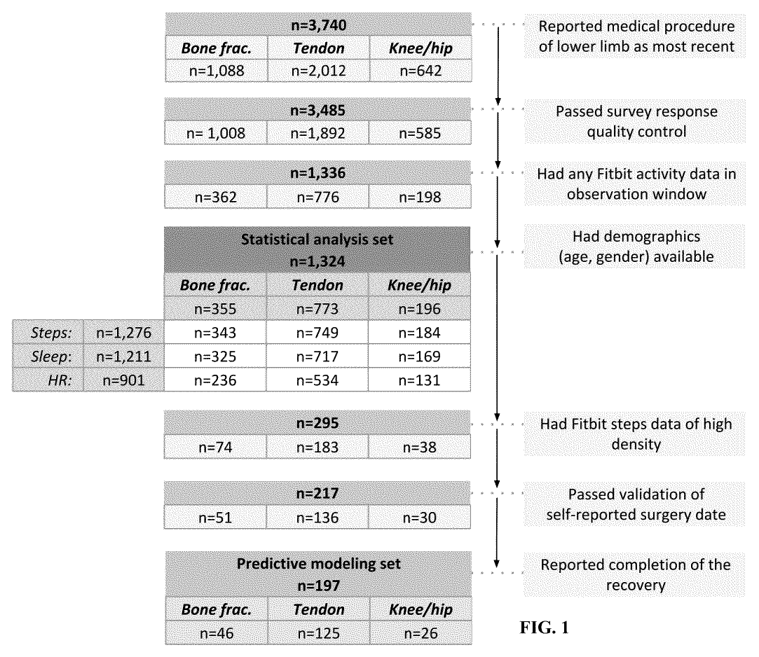

The participants' filtering process is illustrated in FIG. 1. From the initial dataset, participants who had multiple unique answers to questions about the most recent procedure type, or recovery time, or who provided an implausible recovery time label were filtered out (for example, reported recovery time of “3-5 months” where procedure date was less than 3 months from the survey date). The resulting data set consisted of 3,485 participants.

Next, with the participants' permission, their activity datasets were linked for the time window from 182 days (26 weeks) before to 182 days after the self-reported surgery date. To ensure consistency in data quality across the participants, only participants who had any Fitbit device data available in the observation window (n=1,336) were kept. Fitbit devices have been validated and reported as reliable for capturing steps, heart rate, and sleep data; these three data modalities were used to get daily aggregates of various activity and behavioral statistics (see details in Supplementary Note 2). All three modalities are known to be of relevance to post-surgical recovery.

Further, only participants for which age and gender data were available (n=1,324) were kept. Most of the participants had steps (n=1,276) and sleep (n=1,211) data available, fewer participants had heart rate data (n=901). At this point, no participant exclusion criterion due to missing data was applied; data missingness in statistical analysis part of this work is addressed by the choice of a modeling approach, as described below.

Prediction of recovery time required further data filtering to ensure higher data density on a participant-level so that a prediction could be made for each individual. This was achieved by restricting the data sets to participants who (a) did not have continuous periods of missing steps data longer than 28 days, and (b) had at least 50% of observation window days with steps data available. Data coverage in the full statistical analysis data sample (n=1,324) and filtered sample (n=295) is illustrated in Supplementary Note 3.

In order to ensure maximal data quality in the reporting of surgery dates, cases with high likelihood of mis-reporting were systematically identified using a change point detection methodology. This approach was adapted from; a function was fit based on the cohort-level model, and excluded instances where the function strongly fit, but the self-reported and function-reported surgery date were more than 28 days apart. The process is described in more detail in Supplementary Note 4. After applying the rule, n=217 out of 295 participants remained. Finally, only participants who reported completion of the recovery were kept. The final predictive modeling sample had n=197 participants.

### Statistical Modeling of Wearable Person-Generated Health Data (PGHD)

To estimate the impact of medical procedure on steps count, heart rate and sleep, the statistical analysis focused on three activity features: total number of steps, 95th percentile heart rate, and sleep efficiency (the proportion of minutes asleep of the total time in bed) during the main sleep. The baseline time period was defined as weeks from 26 weeks before (the earliest week in observation window) to 13 weeks before the surgery; the upper limit of 13 weeks before the surgery was chosen in order to account for potential cases of relatively long time from injury to surgery (average of 13 weeks of time from injury to surgery was reported in patients with chronic Achilles tendon rupture, where more than half of the cases had tendon rupture after failure of conservative treatment). In all visualizations in the manuscript, “week 0” label denotes a 7 days-long period starting on a self-reported surgery day. Daily activity measurements were modeled with a linear mixed effect model (LMM), fitting a separate model for each activity feature and surgery type subcohort. The outcome was defined as the participant- and day-specific activity measurement. The baseline period and each week in range from 12 before to 26 after the surgery were represented by an indicator variable. The model was adjusted for fixed effects of age, age and relative week interaction, gender, month of the year, weekend day vs. weekday, and participant-specific random effects (baseline activity and weekend day vs. weekday).

To further estimate trajectories of activity across time of recovery groups, the above model was extended by adding indicator variables for self-reported recovery time groups and for recovery group and relative week interaction.

The choice of using day-level activity measurements and employing LMM with participant-specific intercept can avoid a need to enforcing minimal data coverage or performing missing data imputation. Importantly, by including participants with data missingness, statistical power was increased and biasing in population-level estimates of activity was avoided.

### Prediction of Self-Reported Recovery Times

To demonstrate utility of wearable PGHD in predicting long-term trajectories of mobility recovery, the experiment was designed to evaluate performance of classifying self-reported recovery time labels. The machine learning task setup is described in more detail in Supplementary Note 5. In short, the model's performance was compared in six scenarios which differed in assumed availability of PGHD from wearable sensors: (1) no post-operative, no pre-operative, (2) no post-operative, 6 months (full) pre-operative, (3) 4 weeks post-operative, no pre-operative, (4) 4 weeks post-operative, 6 months pre-operative, (5) 6 months (full) post-operative, no pre-operative, (6) 6 months post-operative, 6 months pre-operative; in each (1)-(6) case, demographics (age, gender) information was used.

Due to relatively small sample sizes for bone fracture and knee/hip replacement surgery predictive data sets (n=46 and 26, respectively; see Table 1), the experiment was narrowed down to analyzing the tendon/ligament surgery group only (n=125), and the task was cast as a binary classification of a participant into a faster (“0-2 months”; coded as negative case) and slower (“>=3 months”; coded as positive case) track of mobility recovery. The classification models were trained with the Extreme Gradient Boosting (XGBoost) algorithm and evaluated in the 100-repeat holdout procedure. Alternative algorithms, including random forest with data imputation and feature preselection, and LASSO logistic regression were explored in preliminary stages of analysis and they did not yield performance results better than XGBoost (data not shown). Area under the ROC curve (AUROC) and area under the precision-recall curve (AUPRC) values, obtained on holdout test set across the 100 repetitions, are reported.

### Results

### Study Participants

Table 1 shows a summary of participants demographics and self-reported recovery time for statistical modeling sample (n=1,324) and predictive modeling sample (n=197). Data are summarized for whole sample cohorts (“All”) and for respective strata by surgery type. Participants included in the statistical analysis sample were predominantly female (84%), white or Caucasian (85%), college educated (62%), and young to middle-aged (mean [sd] 36.2 [12.9] years), closely in line with distribution skewness we observed for the whole user base of the Achievement platform (77% female, 88% white or Caucasian, mean age 33 years). The mean age varied across the surgery type sub-cohorts, from 32.9 in bone fracture surgery to 47.7 in knee/hip joint replacement surgery sub-cohort; for comparison, the average age for total hip arthroplasty and total knee arthroplasty patients were reported equal 65 years and 67 years, respectively. The most common self-reported time of recovery fell between 1 and 5 months for bone fracture and knee/hip replacement surgery, and from 1 to 12 months for tendon/ligament repair surgery.

Demographic data summaries for participants included in the predictive modeling sample follow closely the distribution of the analysis data sample. The percentages of self-reported time groups changed mostly due to the fact this sample excluded individuals who have not reported completion of the recovery.

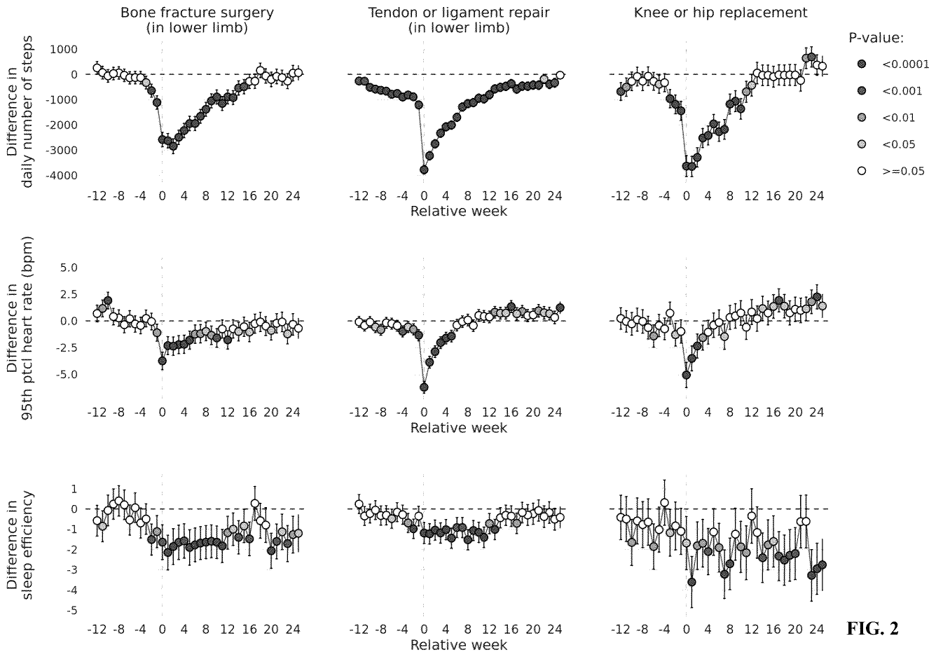

FIG. 2 summarizes the resulting cohort-level model fit, showing, for each surgery type, changes relative to baseline for representative features from step, heart rate and sleep data (daily step count, 95th percentile heart rate and sleep efficiency, respectively) for weeks from 12 before to 26 after the surgery. The trajectories are shown for a “typical” cohort individual (female at age 40, with average baseline activity level among otherwise similar ones). Model-estimated values of activity are also summarized in Table 3 in Supplementary Note 8.

At baseline, the estimated average daily measurement values varied very slightly across three surgery type subcohorts and equal: 8900, 8905, and 8815 for daily sum of steps, 103.9, 102.9, and 103.8 for 95th percentile of heart rate (bpm), 60.4, 57.6, and 57.7 for sleep efficiency—for three surgery type subcohorts (bone fracture repair, tendon or ligament repair/reconstruction, knee or hip joint replacement), respectively. As expected, all surgeries resulted in significant changes in activity, typically reducing daily step counts by 3000 to 4000 steps in the week following surgery, returning to near baseline levels over 8 to 12 weeks. All surgeries also resulted in reductions in submaximal heart rate which generally returned to baseline levels within 4 to 8 weeks and reductions in sleep efficiency which remained throughout the 12 weeks post-surgery. Activity and heart rate data were generally observed to be less variable than sleep data, possibly due to poorer nighttime data coverage and relatively low accuracy of current models for estimating sleep metrics from consumer wearables.

In addition to these general similarities, patterns were also observed that distinguished the three surgery groups and which correspond to distinct best practices. For example, significant pre-surgical reduction in steps sum and heart rate levels was seen in the 2 to 3 weeks prior to bone fracture surgeries, whereas for tendon and ligament surgeries this reduction was already apparent 8 to 10 weeks prior to surgery and for knee or hip replacement the reduction was stronger (more than 1000 steps) and observable 3 to 4 weeks prior to surgery. Distinct post-surgical recovery trajectories were also observed, for example, the effect of bed rest in bone fracture and joint replacement surgeries was visible immediately post surgery, while tendon/ligament repair surgery patients recovered to baseline activity more slowly than the two other groups, which agrees with a slightly higher proportion of self-reported “6-12 months” time of recovery for this group (see Table 1). To confirm the validity of the model, the known effect of age on recovery trajectories was captured (see Supplementary Note 6).

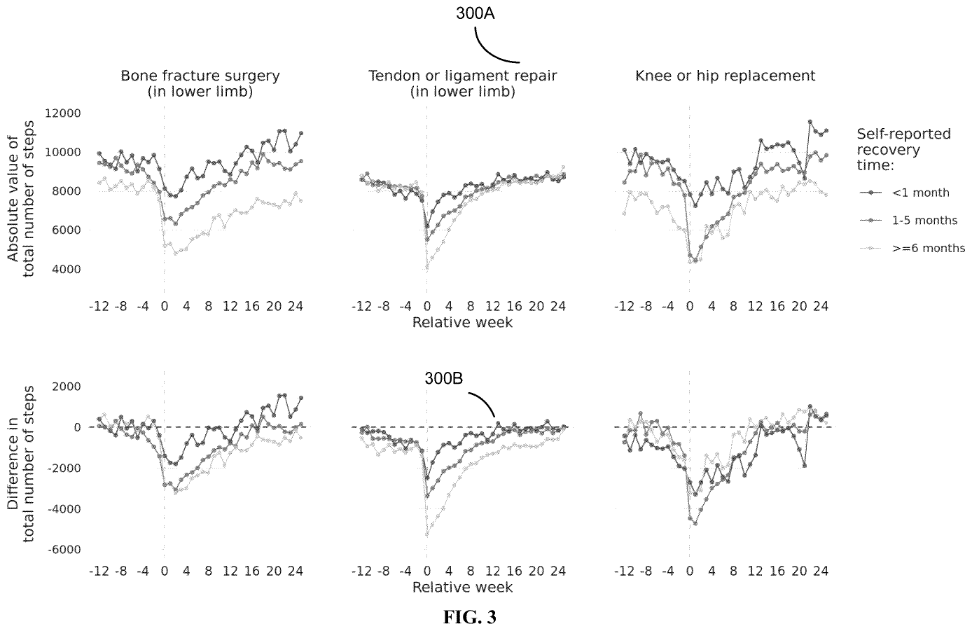

To verify that PGHD from wearable sensors can capture differences in activity across recovery groups, an extended statistical model (see: Methods) was used. FIG. 3 shows estimated average trajectories of daily number of steps across three self-reported recovery time groups, across the three lower limb surgeries. Values are shown for a “typical” cohort individual (female at age 40, with average baseline activity level among similar ones). The upper panel shows absolute activity (steps) values, the bottom plots panel shows change with respect to the model-estimated baseline. In the 1-4 weeks post-operative period, absolute values of activity distinguish the recovery groups, especially for bone fracture and tendon/ligament repair groups. In some embodiments, there is a complementary signal in the trajectory of relative change compared to the baseline, particularly for the tendon/ligament repair subcohort, where differences between the recovery time groups were visible both before and after the surgery. For the knee/hip replacement surgery sub-cohort (the smallest subcohort), relatively higher variability of fitted values was observed; the resulting patterns may have possibly represented a mixture of different knee and hip replacement procedures' effects which cannot be disentangled based on the survey conducted.

Model-estimated values of activity are also summarized in Table 4 in Supplementary Note 8. For completion, activity trajectories estimated across two and across four self-reported recovery time groups are included in Supplementary Note 7.

### Wearable PGHD can be Used to Predict Recovery Trajectories

Table 2 summarizes the results of the experiment to discriminate participants who self-reported faster (“0-2 months”) versus slower (“>=3 months”) functional recovery trajectory, across six scenarios in which different data availability was assumed: demographic data only; individual baseline data only; 1-month post-surgery with and without an individual baseline; 6 months post-surgery with and without individual baseline. The analysis focused on the tendon or ligament surgery group (n=125) as the bone fracture (n=46) and knee/hip replacement (n=26) groups were too small to robustly train and test a predictive model.

Demographic variables (age, gender) themselves were not discriminative between faster and slower recovery track patients, attaining median AUROC of 0.489 (mean 0.473, standard deviation (sd) 0.108; see Table 2). This aligns to high demographic similarity between the recovery groups, for example in the tendon/ligament surgery group, the sample mean of age was very similar in the faster and slower recovery tracks, 36.6 (sd=10.9) and 35.9 (sd=11.3), respectively.

In the 4 weeks post-operative scenarios, the scenario with pre-operative activity data available attained higher AUROC (median=0.734, mean=0.724, sd=0.095) than in the scenario without pre-operative data (AUROC median=0.701, mean=0.705, sd=0.089).

Compared to 4 weeks post-operative scenarios, the 6 months post-operative scenarios yielded results slightly worse when pre-operative activity data were available (median=0.721, mean=0.71, sd=0.096) and slightly better without pre-operative activity data (median=0.716, mean=0.712, sd=0.084).

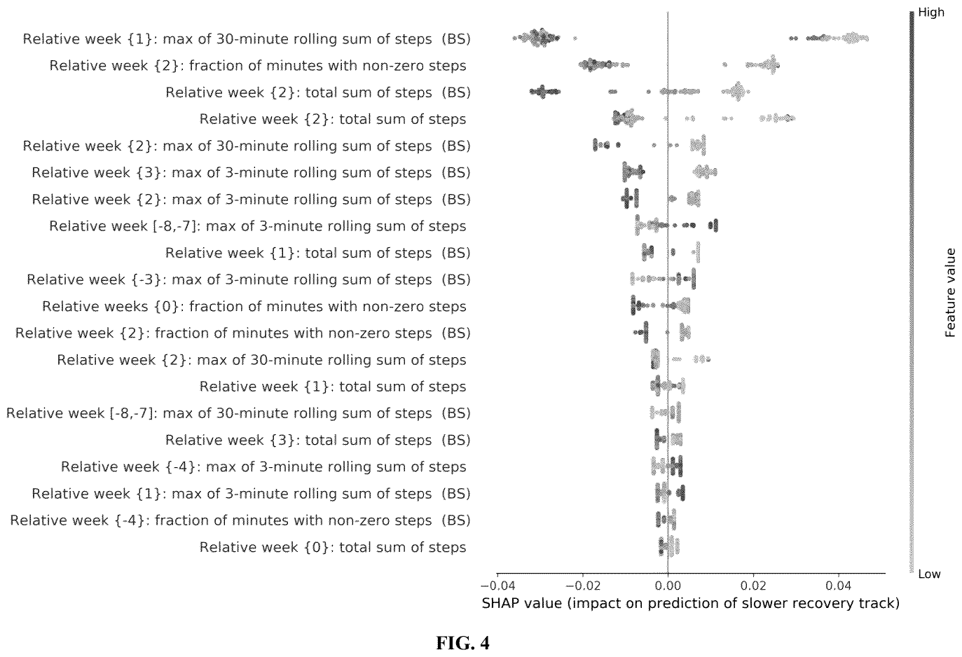

The features relative to baseline and those calculated from weeks immediately around the surgery were observed to be particularly important in driving the predictive power (see FIG. 4). Taken together, these results suggest that 4 weeks post-operative activity data already carry substantial information predictive of a patient's long-term recovery, and that the discriminative power of a model using 4 weeks post-operative activity data may be improved when pre-operative data were available.

### Discussion/Conclusion

Functional recovery trajectories can be accurately modeled based on data from consumer wearable devices describing everyday function from up to 6 months prior to surgery to 6 months post-surgery. Similarly, typical recovery trajectories from different types of surgery can be distinguished, for example the 2-4 weeks of immobilization following bone fracture surgery, versus immediate remobilization of patients following tendon surgery. This model was supported using the known impact of age on functional recovery. Additionally, retrospective, recovery trajectories are clearly differentiated in terms of recovery trajectories, for example by the “depth” of functional limitation immediately post-surgery. Groups can additionally be differentiated based on pre-surgery, long-term baseline function and functional decreases immediately prior to surgery.

Prediction of long-term outcomes is highly important because early intervention, for example increasing exercise, is hypothesized to improve recovery outcomes. Indeed, higher levels of activity prior to surgery can correspond with better functional recovery post-surgery. The accurate prediction of outcomes is often not possible, as pre-surgery risk factors and demographics, without any functional baseline data, do not provide sufficient predictive power, for example 2-year risk of knee replacement revision. Passively collected, consumer-grade wearable data can provide baseline data to accurately predict long-term recovery trajectories. Furthermore, such predictions can be made only 1 month after surgery, early enough to inform alterations to physiotherapy regimes, for example specific targeting of “prehabilitation.” Recent work has also shown that this approach may have value in other therapeutic interventions, for example in oncology.

The data used to train the machine learning model is primarily based on self-reported dates and recovery times. In other embodiments, data to train the machine learning model may be extracted automatically by other sources, including electronic health records (HER), claims data, and from other sources, upon consent of the individual. Data used is conservatively collected to ensure maximal quality, in part enabled by the large scale of data collection. In other embodiments, data can be collected and used from a wider range of consumer devices.

In some implementations, adding more specific information about causes for surgical intervention may prevent further clustering or data analysis without.

### FIGURE LEGENDS

FIG. 1 illustrates study participants' filtering process. Flow chart demonstrates number of participants across three lower limb surgery types: surgery to repair a bone fracture (“Bone frac.”), tendon or ligament repair/reconstruction surgery (“Tendon”), or knee or hip joint replacement surgery (“Knee/hip”).

FIG. 2 illustrates changes in activity features in subsequent weeks from week 12 before to week 26 after the surgery compared to average value in the baseline period (from week 26 to week 13 before the surgery). Horizontal plot panels correspond to three daily features: total number of steps, 95th percentile heart rate, and sleep efficiency during the main sleep. Vertical plot panels correspond to three lower limb surgery types: bone fracture, tendon or ligament repair, and knee or hip replacement. The colors and error bars correspond to p-value value bin and 95% confidence interval of model coefficient estimate for an effect of a relative week compared to baseline, respectively. The “week 0” label (x-axis) denotes a 7 days-long period starting on a self-reported surgery day.

FIG. 3 illustrates plots that show estimated trajectories of daily number of steps of subjects across three self-reported recovery time groups in subsequent weeks from 12 weeks before to 26 weeks after the surgery. The upper plots show absolute values of activity, the bottom plots show activity with respect to the model-estimated baseline. Vertical plot panels correspond to three lower limb surgery types: bone fracture, tendon or ligament repair, and knee or hip replacement. The color of a point/line corresponds to the self-reported recovery time group. The “week 0” label (x-axis) denotes a 7 day-long period starting on a self-reported surgery day.

FIG. 4 illustrates SHapley Additive explanations (SHAP) obtained from hand-tuned XGBoost model fitted to data of all participants in the tendon/ligament surgery group, assuming 4 weeks post-operative and 6 months pre-operative availability of PGHD from wearable sensors. The SHAP values are shown for the top 20 most impactful predictors. The suffix “(BS)” denotes predictors defined as a ratio of value derived from a particular week(s) period to value derived from the baseline period.

TABLE 1
Participants’ demographics and self-reported recovery time for statistical modeling sample
and predictive modeling sample. Data are summarized for the whole
sample cohort (“All”) and by strata by lower limb surgery types: surgery to repair a bone fracture (“Bone frac.”),
tendon or ligament repair/reconstruction surgery (“Tendon”), or knee or hip joint replacement surgery (“Knee/hip”).
Age at the time of procedure was estimated based on
information from a patient ID-linked survey at a different time point than the medical event survey.

| | Statistical modeling set | | | | | | | | |
|---|---|---|---|---|---|---|---|---|---|
| | | | | | | | Knee/ | | |
| | All | | Bone frac. | | Tendon | | hip | | |
| | n = 1,324 | | n = 355 | | n = 773 | | n = 196 | | |
| Gender | | | | | | | | | |
| Female | 1,117 | (84%) | 307 | (86%) | 648 | (84%) | 162 | (83%) | |
| Male | 203 | (15%) | 47 | (13%) | 122 | (16%) | 34 | (17%) | |
| Other | 4 | (<1%) | 1 | (<1%) | 3 | (<1%) | 0 | (0%) | |
| Race | | | | | | | | | |
| White or | 1,119 | (85%) | 302 | (85%) | 649 | (84%) | 168 | (86%) | |
| Caucasian | | | | | | | | | |
| Black or | 52 | (4%) | 15 | (4%) | 30 | (4%) | 7 | (4%) | |
| African | | | | | | | | | |
| American | | | | | | | | | |
| Hispanic or | 61 | (5%) | 14 | (4%) | 39 | (5%) | 8 | (4%) | |
| Latino | | | | | | | | | |
| Other | 45 | (3%) | 10 | (3%) | 26 | (3%) | 9 | (5%) | |
| Unavailable | 47 | (4%) | 14 | (4%) | 29 | (4%) | 4 | (2%) | |
| Age | | | | | | | | | |
| mean (sd) | 36.2 | (12.9) | 32.9 | (11.1) | 34.9 | (11.7) | 47.7 | (14.4) | |
| median | 34 | [18, | 31 | [18, | 33 | [18, | 51 | [18, | |
| [min, max] | | 77] | | 70] | | 70] | | 77] | |
| Education | | | | | | | | | |
| Doctorate, | 29 | (2%) | 5 | (1%) | 19 | (2%) | 5 | (3%) | |
| MD | | | | | | | | | |
| Graduate | 236 | (18%) | 58 | (16%) | 146 | (19%) | 32 | (16%) | |
| degree | | | | | | | | | |
| College | 515 | (39%) | 133 | (37%) | 317 | (41%) | 65 | (33%) | |
| degree | | | | | | | | | |
| (AS or BS) | | | | | | | | | |
| Some college | 311 | (23%) | 87 | (25%) | 183 | (24%) | 41 | (21%) | |
| Trade or | 77 | (6%) | 23 | (6%) | 33 | (4%) | 21 | (11%) | |
| vocational | | | | | | | | | |
| training | | | | | | | | | |
| High school | 107 | (8%) | 31 | (9%) | 48 | (6%) | 28 | (14%) | |
| diploma/GED | | | | | | | | | |
| No high | 9 | (1%) | 5 | (1%) | 4 | (1%) | 0 | (0%) | |
| school | | | | | | | | | |
| diploma | | | | | | | | | |
| Unavailable | 40 | (3%) | 13 | (4%) | 23 | (3%) | 4 | (2%) | |
| Recovery | | | | | | | | | |
| time | | | | | | | | | |
| <1 month | 260 | (20%) | 61 | (17%) | 164 | (21%) | 35 | (18%) | |
| 1-2 months | 257 | (19%) | 80 | (23%) | 137 | (18%) | 40 | (20%) | |
| 3-5 months | 292 | (22%) | 96 | (27%) | 151 | (20%) | 45 | (23%) | |
| 6-12 months | 209 | (16%) | 38 | (11%) | 142 | (18%) | 29 | (15%) | |
| 1 year | 28 | (2%) | 8 | (2%) | 17 | (2%) | 3 | (2%) | |
| or longer | | | | | | | | | |
| I never fully | 33 | (2%) | 8 | (2%) | 20 | (3%) | 5 | (3%) | |
| recovered | | | | | | | | | |
| I'm still | 245 | (19%) | 64 | (18%) | 142 | (18%) | 39 | (20%) | |
| recovering | | | | | | | | | |
| | | Predictive modeling set | | | | | | | |
| | | | | Bone | | | | Knee/ | |
| | | All | | frac. | | Tendon | | hip | |
| | | n = 197 | | n = 46 | | n = 125 | | n = 26 | |
| | Gender | | | | | | | | |
| | Female | 169 | (86%) | 41 | (89%) | 106 | (85%) | 22 | (85%) |
| | Male | 28 | (14%) | 5 | (11%) | 19 | (15%) | 4 | (15%) |
| | Other | 0 | (0%) | 0 | (0%) | 0 | (0%) | 0 | (0%) |
| | Race | | | | | | | | |
| | White or | 168 | (85%) | 37 | (80%) | 110 | (88%) | 21 | (81%) |
| | Caucasian | | | | | | | | |
| | Black or | 5 | (3%) | 1 | (2%) | 4 | (3%) | 0 | (0%) |
| | African | | | | | | | | |
| | American | | | | | | | | |
| | Hispanic or | 8 | (4%) | 2 | (4%) | 4 | (3%) | 2 | (8%) |
| | Latino | | | | | | | | |
| | Other | 6 | (3%) | 3 | (7%) | 2 | (2%) | 1 | (4%) |
| | Unavailable | 10 | (5%) | 3 | (7%) | 5 | (4%) | 2 | (8%) |
| | Age | | | | | | | | |
| | mean (sd) | 37.3 | (11.6) | 34.2 | (8.9) | 36.1 | (11.1) | 48.4 | (12.4) |
| | median | 35 | [18, | 32 | [19, | 34 | [18, | 49 | [24, |
| | [min, max] | | 71] | | 53] | | 64] | | 71] |
| | Education | | | | | | | | |
| | Doctorate, | 4 | (2%) | 0 | (0%) | 4 | (3%) | 0 | (0%) |
| | MD | | | | | | | | |
| | Graduate | 33 | (17%) | 8 | (17%) | 22 | (18%) | 3 | (12%) |
| | degree | | | | | | | | |
| | College | 89 | (45%) | 24 | (52%) | 58 | (46%) | 7 | (27%) |
| | degree | | | | | | | | |
| | (AS or BS) | | | | | | | | |
| | Some college | 42 | (21%) | 7 | (15%) | 26 | (21%) | 9 | (35%) |
| | Trade or | 8 | (4%) | 4 | (9%) | 3 | (2%) | 1 | (4%) |
| | vocational | | | | | | | | |
| | training | | | | | | | | |
| | High school | 13 | (7%) | 1 | (2%) | 8 | (6%) | 4 | (15%) |
| | diploma/GED | | | | | | | | |
| | No high | 1 | (1%) | 0 | (0%) | 1 | (1%) | 0 | (0%) |
| | school | | | | | | | | |
| | diploma | | | | | | | | |
| | Unavailable | 7 | (4%) | 2 | (4%) | 3 | (2%) | 2 | (8%) |
| | Recovery | | | | | | | | |
| | time | | | | | | | | |
| | <1 month | 37 | (19%) | 6 | (13%) | 26 | (21%) | 5 | (19%) |
| | 1-2 months | 51 | (26%) | 16 | (35%) | 29 | (23%) | 6 | (23%) |
| | 3-5 months | 52 | (26%) | 15 | (33%) | 28 | (22%) | 9 | (35%) |
| | 6-12 months | 53 | (27%) | 7 | (15%) | 41 | (33%) | 5 | (19%) |
| | 1 year | 4 | (2%) | 2 | (4%) | 1 | (1%) | 1 | (4%) |
| | or longer | | | | | | | | |
| | I never fully | 0 | (0%) | 0 | (0%) | 0 | (0%) | 0 | (0%) |
| | recovered | | | | | | | | |
| | I'm still | 0 | (0%) | 0 | (0%) | 0 | (0%) | 0 | (0%) |
| | recovering | | | | | | | | |

TABLE 2
Performance of predictive models in the task of discriminating
participants between a faster (“0-2 months”) and slower
(“>=3 months”) track of mobility recovery. Results
are shown across six experiment scenarios in which different
data availability was assumed (in each case, age and gender
demographic information was used), and across surgery types considered.

| Experiment scenario | AUROC | | AUPRC | |
|---|---|---|---|---|
| (post-/pre-operative | mean | median | mean | median |
| wearable PGHD | (sd) | [min, max] | (sd) | [min, max] |
| availability) | Tendon or ligament repair/reconstruction surgery | | | |
| (1) no post-op, | 0.473 | 0.489 | 0.569 | 0.563 |
| no pre-op | (0.108) | [0.114, 0.669] | (0.059) | [0.428, 0.727] |
| (2) no post-op, | 0.497 | 0.498 | 0.596 | 0.596 |
| 6 m pre-op | (0.096) | [0.266, 0.734] | (0.068) | [0.453, 0.741] |
| (3) 4 wk post-op, | 0.705 | 0.701 | 0.784 | 0.799 |
| no pre-op | (0.089) | [0.510, 0.929] | (0.076) | [0.598, 0.947] |
| (4) 4 wk post-op, | 0.724 | 0.734 | 0.795 | 0.800 |
| 6 m pre-op | (0.095) | [0.442, 0.942] | (0.077) | [0.576, 0.960] |
| (5) 6 m post-op, | 0.712 | 0.716 | 0.798 | 0.806 |
| no pre-op | (0.084) | [0.542, 0.929] | (0.067) | [0.628, 0.937] |
| (6) 6 m post-op, | 0.710 | 0.721 | 0.786 | 0.791 |
| 6 m pre-op | (0.096) | [0.435, 0.942] | (0.080) | [0.542, 0.960] |

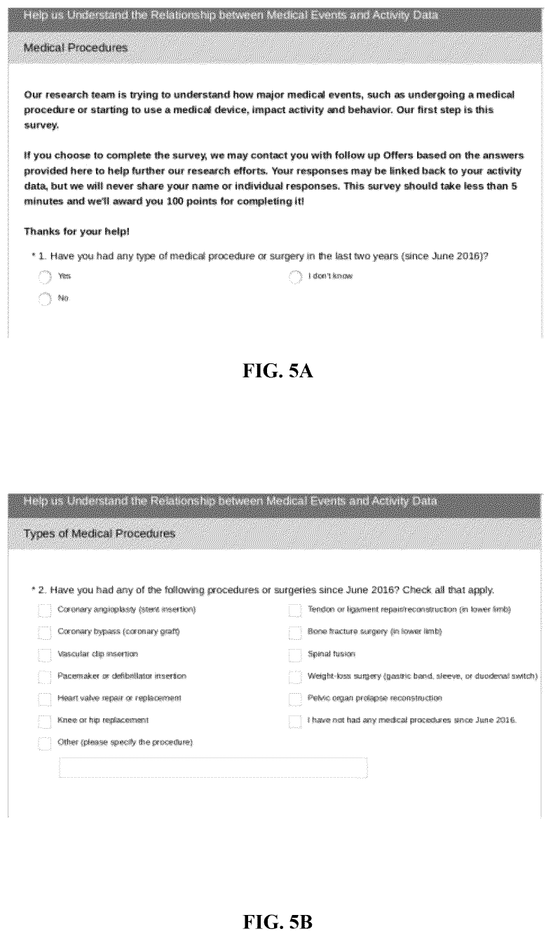

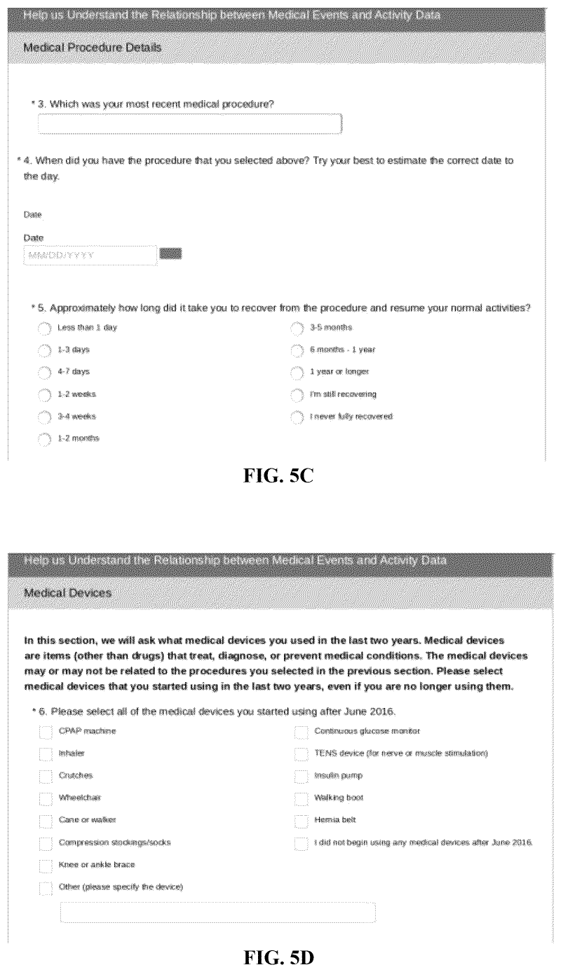

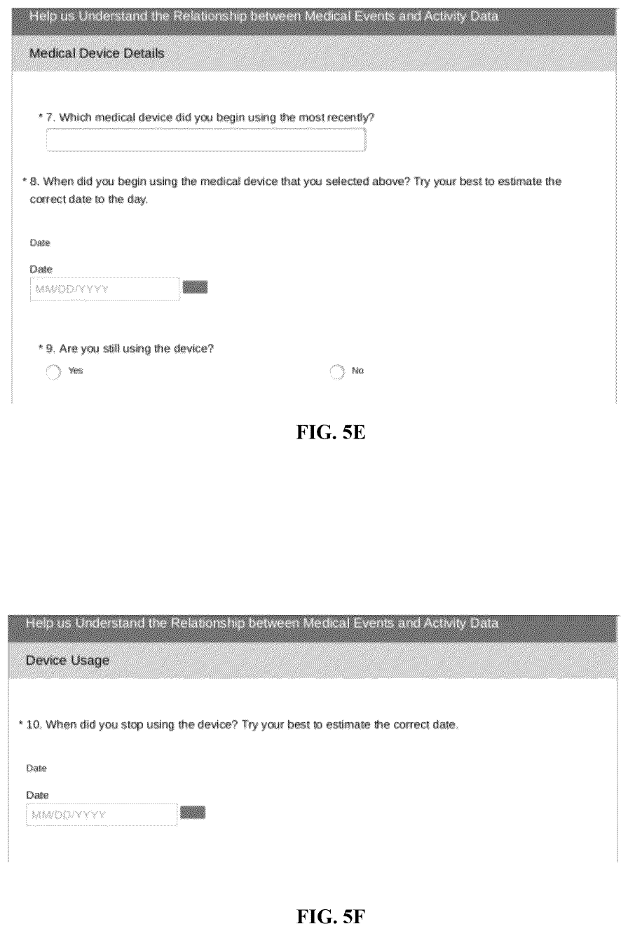

Supplementary Note 1: Survey (FIG. 5A-F). FIGS. 5A-5 illustrates snapshots of the full survey deployed to users of the application. The survey asked about medical procedures the members have undergone in the 2 years prior to taking the survey.

Supplementary Note 2: Processing of steps, heart rate, and sleep data. Fitbit-collected data of steps, heart rate, and sleep were used to get daily aggregates of activity statistics. A part of daily activity features used in this work (sleep efficiency) were accessed from the public Fitbit application programming interface, whereas others were derived from the minute-level intraday activity data (total number of steps, fraction of minutes with >0 steps, maximum of 3- and 30-minute rolling steps sum, 95th percentile heart rate). Selected step daily features (total number of steps, maximum of 3- and 30-minute rolling steps sum) were winsorized at respective 0.999-th quantiles. The daily sleep efficiency feature, ranging originally 0-100 (mean=90.7, sd=11.4), was transformed with a log-based function to handle its high positive skewness, resulting in a (modified) efficiency feature ranging 0-100 (mean=56.8, sd=16.7).

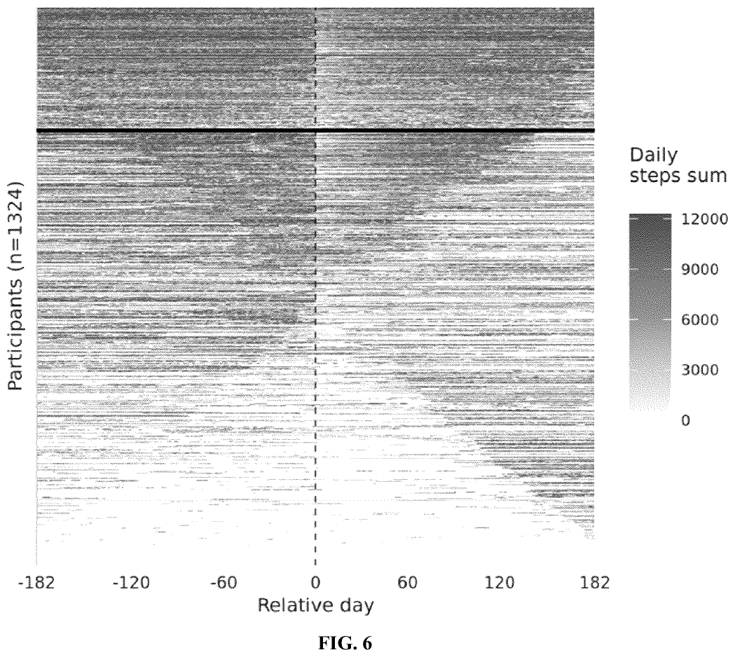

Supplementary Note 3: Data coverage (FIG. 6). FIG. 6 illustrates a plot showing step data coverage in a statistical analysis sample (n=1,324). The heatmap color corresponds to the daily number of steps (winsorized at 12,000 for visualization purposes) across days relative to self-reported surgery date (x-axis) in the observation window from 182 days before to 182 days after the surgery. The solid black horizontal line separates participants (n=295) who passed a step data density requirement for use in the experiment.

Supplementary Note 4: Data preparation for machine learning. To ensure maximal data quality in the reporting of surgery dates, cases with high likelihood of misreporting were systematically identified using a change point detection methodology:

At each time t of the daily number of steps time-series of length T, a four-piecewise function was fitted with the main change point located at t and the remaining function components optimized to minimize the fit residuals. The function's shape was restricted to represent expected post-surgery activity pattern, but flexible enough to account for various lengths of recovery and signal strength, or even no signal at all (see the figure below, part (a)). The likelihood Lₜ of the main change point being at time t was quantified by using a (standardized) difference between residuals e from single constant fit and residuals uₜ from fitted four-piecewise function with k parameters: Lt=(Σe²−Σuₜ²)/(Σuₜ²)/(T−k). In the figure below, part (b) shows an exemplary trajectory of Lₜ values for one participant. Time point t=tₘₐₓ that maximizes Lₜ for a participant was defined as an algorithm-identified surgery date.

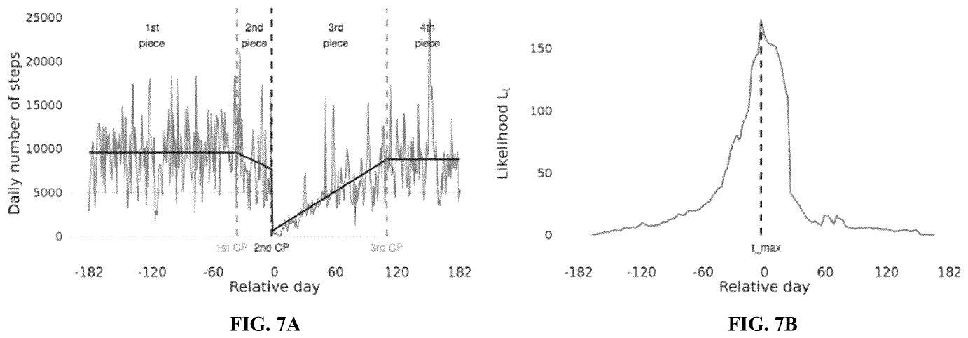

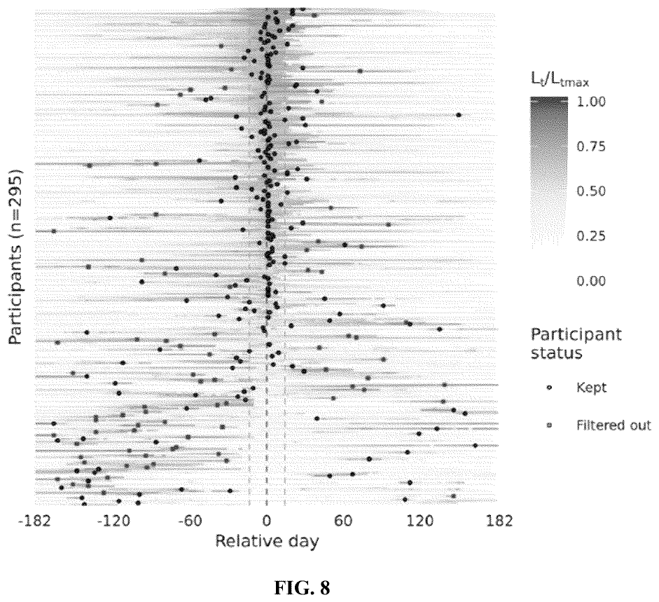

FIG. 7A provides an illustration of a four-piecewise fit used in change point (CP) detection procedure: (1) 1st piece: a constant, (2) 1st CP, (3) 2nd piece: a linear function with negative slope joined with 1st piece, or a constant same as 1st piece, (3) 2nd CP: the main CP located at a fixed time point t, (4) 3rd piece: a linear function with positive slope joined with 4th piece, or a constant same as 4th piece, (5) 3rd CP, (6) 4th piece: a constant. In the procedure, at each fixed time point t, the 2nd CP is fixed at t, and the remaining components of the four piecewise fit are optimized to reduce the fit residuals. FIG. 7B illustrates an exemplary trajectory of the likelihood Lt. Here, t=−3 maximizes Lt; the fit that corresponds to 2nd CP located at t=−3 is shown in figure left (a).

Finally, the maximum likelihood value, L_{t_max}, was used to propose a heuristic rule: (a) if best fit signal is strong (L_{t_max} statistic above 30) and self-reported and algorithm-identified surgery date are more than 28 days apart—a participant is filtered out; (b) otherwise—participant is kept. After applying the rule, n=217 out of 295 participants were kept. The figure below shows normalized likelihood trajectories, (Lₜ/L_{t_max}), together with algorithm-identified surgery date for kept and rejected participants.

FIG. 8 illustrates a plot showing participants' (normalized) likelihood trajectories, (Lt/Lt_max), of the main change point being at time t across the observation window of 182 days before and 182 days after self-reported surgery time (x-axis).

Supplementary Note 5: Prediction of self-reported recovery times. The experiment was designed to evaluate performance of classifying self-reported recovery time labels. The model's performance was compared in six scenarios which differed in assumed availability of wearable PGHD: (1) no post-operative, no pre-operative, (2) no post-operative, 6 months (full) pre-operative, (3) 4 weeks post-operative, no pre-operative, (4) 4 weeks postoperative, 6 months pre-operative, (5) 6 months (full) post-operative, no pre-operative, (6) 6 months post-operative, 6 months pre-operative; in each (1)-(6) case, demographics (age, gender) information was used.

Due to relatively small sample sizes for bone fracture and knee/hip replacement surgery predictive data sets (n=46 and 26, respectively; see Table 1), the experiment was narrowed down to analyzing the tendon or ligament surgery group only (n=125), and the machine learning task was cast as a binary classification of a participant into a faster (“0-2 months”) and slower (“>=3 months”) track of mobility recovery.

For each participant, a set of predictors was computed based on the four steps-derived daily measurements: total number of steps, fraction of minutes with non-zero steps count, number of steps in max of 3- and 30-minute rolling sum. The predictors were constructed as a measurement aggregate (median) over week(s) of time; the length of aggregation time period varied between one and 14 weeks long depending on distance from surgery date (the closer to the surgery, the higher resolution of the time periods). The aggregation of daily measures into irregular time periods was performed to avoid an extremely large ratio of number of predictors to number of observations while simultaneously making the most use of the data signal available.

In the notation used in this work, “relative week 0” always corresponds to a 7-day-long period that starts at the day of surgery, “relative week 1” lasts from the 7th to 13th day (inclusive) after the surgery, and “relative week −1” lasts from the 7th to 1st day (inclusive) before the surgery, etc. Then, activity measurements collected in relative weeks from −4 to 4 were aggregated over time periods of one week, activity measurements collected in relative weeks from −8 to −5 and from 5 to 8 were aggregated over time periods of two subsequent weeks, activity measurements collected in relative weeks from −12 to −9 and from 9 to 12 were aggregated over time periods of four subsequent weeks, activity measurements collected in relative weeks from −26 to −13 and from 13 to 26 were aggregated over time periods of fourteen weeks, respectively (the relative week 26 was exceptional as it consisted of 1 day only).

These predictors were further standardized to have mean 0 and variance 1 to avoid large differences in the order of values across predictors in the data set.

Also, to reflect participant's activity change w.r.t to the baseline (relative weeks from −26 to −13), additional predictors were defined as a ratio of (a) particular time period-aggregated value to (b) baseline weeks-aggregated value; these predictors were used in modeling only in the scenarios assuming pre-operative data is available. These variables were winsorized at value equal 3.

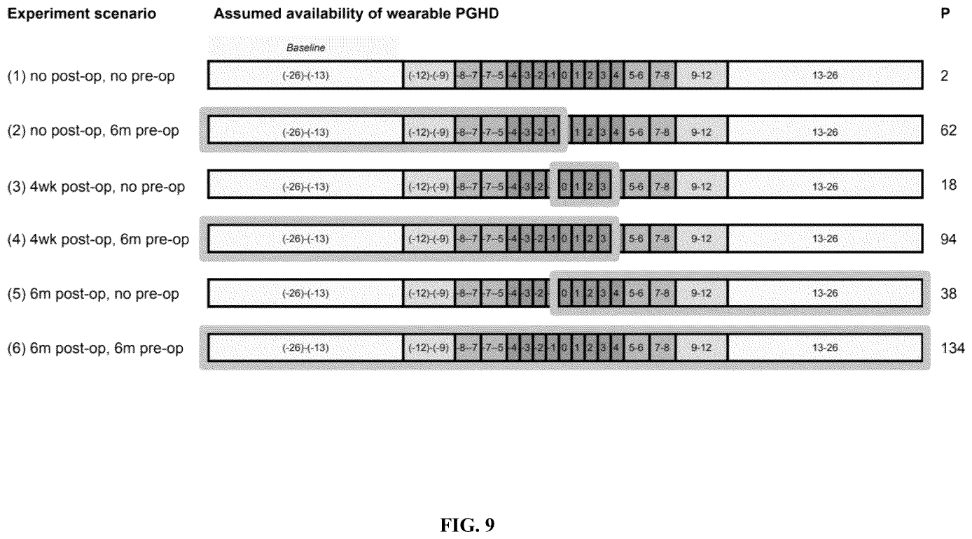

FIG. 9 shows assumed wearable PGHD availability in predictive modeling experiment scenarios (1)-(6). In each scenario, demographics (age, gender) were also used. The black rectangular box grid represents the grouping of relative week(s) into time periods for aggregation of daily activity measurements. The numbers within rectangular blocks denote a range of relative weeks within a certain aggregation time period. Green rectangular box is used to mark the weeks relative to the surgery from which wearable PGHD is assumed available in scenarios (2)-(6). The last column, “P,” summarizes the number of predictors (demographics and activity predictors combined) in each scenario.

The classification models were trained with the Extreme Gradient Boosting (XGBoost) algorithm. The choice of the algorithm was driven by its performance, ability to handle missing data, and interpretability of the results. A 100-repeat holdout procedure was used to estimate out-of-sample generalization of models' classification performance. In each of 100 repetitions, the dataset was split into training and test sets using an 80/20 split that was stratified by the outcome (faster, “0-2 months,” and slower, “>=3 months,” track of mobility recovery). Hyper-parameters were tuned on the training set by comparing AUROC predictive metric aggregated over 20 repetitions of 75/25 split stratified by the outcome; tuning was done by selecting the best combination of the following parameters: number of estimators, learning rate, maximum tree depth, gamma, minimum child weight, subsample proportion, out of 144 combinations considered. Then, the best parameters set was used to train the model on a full training set and to measure predictive performance on the holdout test sample. The predictive performance metric values (AUROC, AUPRC) summarized across 100 repetitions are reported.

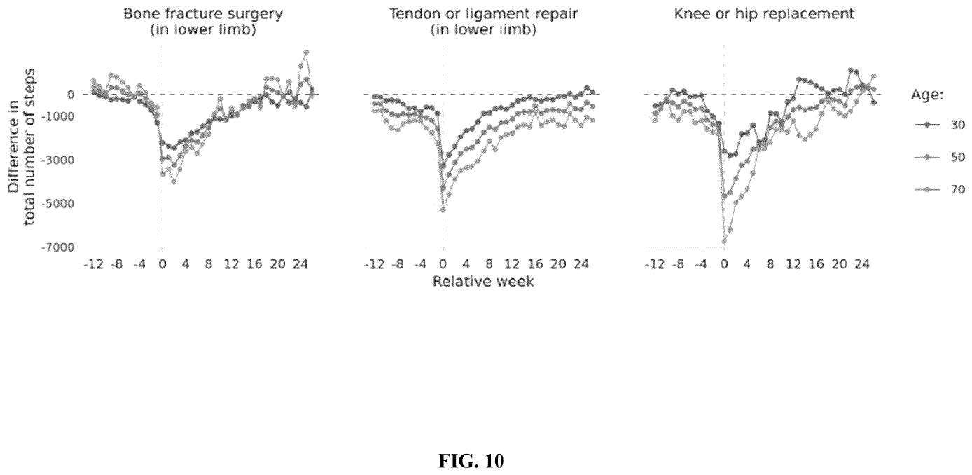

Supplementary Note 6: Impact of age on recovery trajectories. To demonstrate the validity of the cohort-level model, known effects due to age were explored. Increasing age is known to have a strong, negative influence on recovery timelines. The statistical model was therefore used to estimate average recovery trajectories at a range of ages (30, 50 and 70 years old), for an otherwise “typical” individual (female, with average baseline activity level among similar ones). The FIG. 10 describes fitted age-specific trajectories of daily number of steps across the three lower limb surgeries. Clearly, the age effect is demonstrated with higher difference in activity values after surgery compared to respective baseline levels. This effect is pronounced particularly strongly in knee/hip replacement sub-cohort in 1-5 weeks after the procedure; while it is not possible to determine the difference between the cases of knee and hip surgery based on the survey conducted, one can hypothesize that the values fitted for 70-years-old individual represent a higher proportion of hip replacement cases and correspond to a full/almost full immobilization days after the procedure.

FIG. 10 illustrates a daily total number of steps in subsequent weeks from 12 week before to 26 week after surgery compared to average value in the baseline period (weeks from 26 weeks before to 13 weeks before the surgery) for individuals at age 30, 50 and 70 and otherwise “typical” (female, with average baseline activity level among similar ones). Vertical plot panels correspond to three lower limb surgery types: bone fracture, tendon or ligament repair, and knee or hip replacement. The color of a point/line corresponds to the individual's age.

Supplementary Note 7: Trajectories of recovery across self-reported recovery time groups.

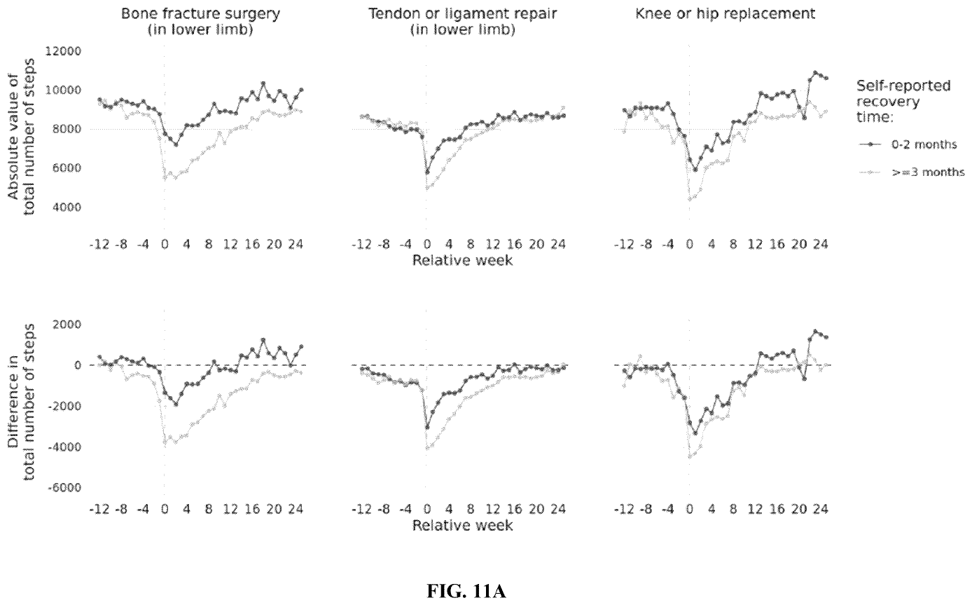

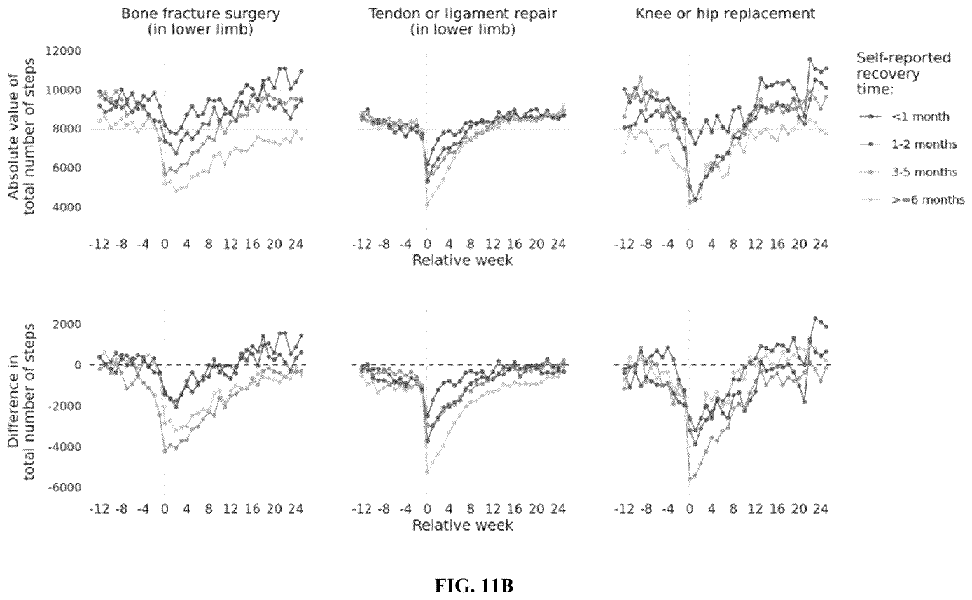

FIG. 11A shows a set of plots illustrating estimated trajectories of daily number of steps across two self-reported recovery time groups in subsequent weeks from week 12 before to week 26 after the surgery. The upper plots demonstrate absolute values of activity, the bottom plots demonstrate change with respect to the model-estimated baseline. Vertical plots correspond to three lower limb surgery types: bone fracture, tendon or ligament repair, and knee or hip replacement.

FIG. 11B shows a set of plots illustrating estimated trajectories of daily number of steps across four self-reported recovery time groups in subsequent weeks from week 12 before to week 26 after the surgery. The upper plots demonstrate absolute values of activity, the bottom plots panel—change with respect to the model-estimated baseline. Vertical plots correspond to three lower limb surgery types: bone fracture, tendon or ligament repair, and knee or hip replacement.

Supplementary note 8: Model-estimated average values of activity daily measurements

TABLE 3
Model-estimated average values of activity daily measurements (daily
number of steps, 95^{th} percentile of heart rate (bpm), sleep efficiency) across three surgery type
subcohorts (bone fracture repair, tendon or ligament repair/reconstruction, knee or hip joint
replacement) and across eight time periods relative to self-reported surgery date: baseline and
relative weeks −4, 0, 4, 8, 12, 16, 20. Relative week “0” was defined as a 7-day-long period that starts
at the day of surgery. Baseline was defined as relative weeks from −26 to −13. Showed are values
estimated for a “typical” cohort individual (female at age 40, with average baseline activity level
among otherwise similar ones) on a “typical” day (weekday, month of May).

| Activity | Surgery | | | | | | | | |
|---|---|---|---|---|---|---|---|---|---|
| daily | type | Time period relative to self-reported surgery date | | | | | | | |
| measurement | subcohort | Baseline | Week −4 | Week 0 | Week 4 | Week 8 | Week 12 | Week 16 | Week 20 |
| Number of | Bone frac. | 8900 | 8765 | 6315 | 6672 | 7512 | 8004 | 8618 | 8695 |
| steps | | | | | | | | | |
| Number of | Tendon | 8905 | 8003 | 5124 | 6823 | 7742 | 8095 | 8533 | 8478 |
| steps | | | | | | | | | |
| Number of | Knee/hip | 8815 | 8483 | 5179 | 6392 | 7632 | 8379 | 8718 | 8786 |
| steps | | | | | | | | | |
| 95th ptcl HR | Bone frac. | 103.9 | 103.4 | 100.1 | 101.7 | 102.9 | 102.1 | 102.8 | 103.0 |
| 95th ptcl HR | Tendon | 102.9 | 101.9 | 96.7 | 101.3 | 103.0 | 103.2 | 104.2 | 103.4 |
| 95th ptcl HR | Knee/hip | 103.8 | 103.1 | 98.8 | 102.8 | 104.2 | 104.7 | 105.2 | 104.9 |
| Sleep | Bone frac. | 60.4 | 59.7 | 58.8 | 58.8 | 58.8 | 59.3 | 58.9 | 58.4 |
| efficiency | | | | | | | | | |
| Sleep | Tendon | 57.6 | 57.4 | 56.4 | 56.6 | 56.1 | 56.9 | 57.3 | 57.4 |
| efficiency | | | | | | | | | |
| Sleep | Knee/hip | 57.7 | 58.1 | 56.1 | 55.6 | 55.0 | 57.4 | 56.1 | 55.5 |
| efficiency | | | | | | | | | |

TABLE 4
Model-estimated average values of activity daily measurement (daily
number of steps) across three surgery type subcohorts (bone fracture repair, tendon or ligament
repair/reconstruction, knee or hip joint replacement), across three self-reported recovery time groups
(<1 month, 1-5 months, >=6 months), and across eight time periods relative to self-reported surgery
date: baseline and relative weeks −4, 0, 4, 8, 12, 16, 20. Relative week “0” was defined as a 7-day-
long period that starts at the day of surgery. Baseline was defined as relative weeks from −26 to −13.
Showed are values estimated for a “typical” cohort individual (female at age 40, with average
baseline activity level among otherwise similar ones) on a “typical” day (weekday, month of May).

| | | Self- | | | | | | | | | |
|---|---|---|---|---|---|---|---|---|---|---|---|
| Activity | Surgery | reported | | Time period relative to self-reported surgery date | | | | | | | |
| daily | type | recovery | | Base- | Week | Week | Week | Week | Week | Week | Week |
| measurement | subcohort | time gr. | | line | −4 | 0 | 4 | 8 | 12 | 16 | 20 |
| Number of | Bone | <1 | month | 9536 | 9679 | 8126 | 8726 | 9509 | 8848 | 10063 | 10108 |
| steps | frac. | | | | | | | | | | |
| Number of | Bone | 1-5 | months | 9386 | 9116 | 6561 | 7047 | 7937 | 8593 | 9462 | 9354 |
| steps | frac. | | | | | | | | | | |
| Number of | Bone | >=6 | months | 8027 | 8188 | 5192 | 5023 | 5783 | 6751 | 7158 | 7310 |
| steps | frac. | | | | | | | | | | |
| Number of | Tendon | <1 | month | 8676 | 7612 | 6189 | 7888 | 8380 | 8335 | 8821 | 8485 |
| steps | | | | | | | | | | | |
| Number of | Tendon | 1-5 | months | 8885 | 8182 | 5518 | 6891 | 7764 | 8182 | 8742 | 8786 |
| steps | | | | | | | | | | | |
| Number of | Tendon | >=6 | months | 9359 | 8099 | 4119 | 6017 | 7549 | 8059 | 8516 | 8408 |
| steps | | | | | | | | | | | |
| Number of | Knee/hip | <1 | month | 10542 | 9590 | 7829 | 7847 | 8987 | 9166 | 10391 | 9448 |
| steps | | | | | | | | | | | |
| Number of | Knee/hip | 1-5 | months | 9177 | 9152 | 4705 | 6185 | 7693 | 8880 | 9381 | 8966 |
| steps | | | | | | | | | | | |
| Number of | Knee/hip | >=6 | months | 7587 | 7221 | 4362 | 5866 | 7213 | 7579 | 7491 | 8436 |

steps

Supplementary note 9: Model summary output. In statistical modeling of wearable PGHD, daily activity measurements were modeled with a linear mixed effect model (LMM), fitting a separate model (model 1) for each activity feature and surgery type subcohort. To further estimate trajectories of activity across time of recovery groups, the statistical model was extended by considering variables for self-reported recovery time groups (model 2—“extended”). Below we define LMM formula notation (common for both model 1 and model 2—“extended”) and report elements of LMM fit summary—variance and correlation components and coefficient estimates—for model 1 and model 2—“extended,” respectively. For the sake of space, we limit the report to one activity feature (daily sum of steps) and one surgery type subcohort (bone fracture surgery).

### LMM Formula Notation:

The LMM formulas and LMM fit summary elements presented below share the following notation for data variables.
y—Numeric variable. Participant- and day-specific activity measurement.
time indic—Factor variable. A week relative to a self-reported surgery date. Takes values: {baseline, −12, −11, . . . , −1, 0, 1, . . . , 26}, where “baseline” is set as reference factor level. Relative week “0” is a 7 days-long time period starting on a self-reported surgery day. “Baseline” is a time period defined as weeks from 26 week before to 13 week before the surgery.
age_centered—Numeric variable. Participant's age, centered at 40 (has value 0 for a 40 years old participant.
gender—Factor variable. Self-reported participant gender. Takes values: {female,male,other}, where “female” is set as reference factor level.
date_isweekend—Factor variable. Flag whether or not a participant- and day-specific activity measurement was collected on a weekend day. Takes values: {0,1}, where “0” is set as reference factor level.
date_years_month—Factor variable. Label for a month of a year. Takes values: {Jan, Feb, . . . , Nov, Dec}, where “May” is set as reference factor level.
user_id—Factor variable. Participant-specific ID.
recovery_gr—Factor variable. Label for self-reported recovery time groups. Takes values: (a) {0-2 months, >=3 months}, where “0-2 months” is set as reference factor level, or (b) {<1 month, 1-5 months, >=6 months}, where “1-5 months” is set as reference factor level, or (c) {<1 month, 1-2 months, 3-5 months, >=6 months} where “1-2 months” is set as reference factor level.

### Linear Mixed Effect Model 1:

| y ~ time_indic * age_centered + gender + date_isweekend + | | | |
|---|---|---|---|
| date_years_month + (1 + date_isweekend \| user_id) | | | |
| Groups | Name | Std. Dev. | Corr. |
| user_id | (Intercept) | 3444.2 | |
| | date_isweekend | 2043.2 | −0.438 |
| Residual | | 4085.2 | |

### Linear Mixed Effect Model 2—“Extended”:

| y ~ time_indic * age_centered + time_indic * recovery_gr + | | | |
|---|---|---|---|
| age_centered + gender + date_isweekend + date_years_month + | | | |
| (1 + date_isweekend \| user_id) | | | |
| Groups | Name | Std. Dev. | Corr. |
| user_id | (Intercept) | 3540.8 | |
| | date_isweekend | 2126.4 | −0.495 |
| Residual | | 4150.7 | |

### Computer Systems

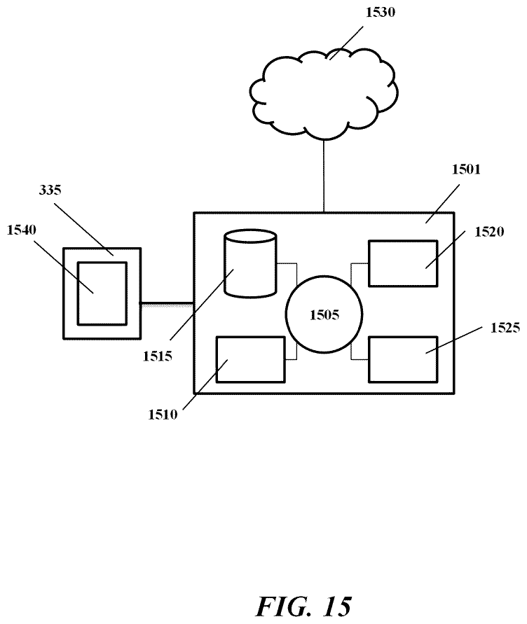

The present disclosure provides computer systems that are programmed to implement methods of the disclosure. FIG. 15 shows a computer system 1501 that is programmed or otherwise configured to predict time to recovery from wearable sensor data. The computer system 1501 can regulate various aspects of predicting time to recovery of the present disclosure, such as, for example, implementing one or more machine learning algorithms. The computer system 1501 can be an electronic device of a user or a computer system that is remotely located with respect to the electronic device. The electronic device can be a mobile electronic device.

The computer system 1501 includes a central processing unit (CPU, also “processor” and “computer processor” herein) 1505, which can be a single core or multi core processor, or a plurality of processors for parallel processing. The computer system 1501 also includes memory or memory location 1510 (e.g., random-access memory, read-only memory, flash memory), electronic storage unit 1515 (e.g., hard disk), communication interface 1520 (e.g., network adapter) for communicating with one or more other systems, and peripheral devices 1525, such as cache, other memory, data storage and/or electronic display adapters. The memory 1510, storage unit 1515, interface 1520 and peripheral devices 1525 are in communication with the CPU 1505 through a communication bus (solid lines), such as a motherboard. The storage unit 1515 can be a data storage unit (or data repository) for storing data. The computer system 1501 can be operatively coupled to a computer network (“network”) 1530 with the aid of the communication interface 1520. The network 1530 can be the Internet, an internet and/or extranet, or an intranet and/or extranet that is in communication with the Internet. The network 1530 in some cases is a telecommunication and/or data network. The network 1530 can include one or more computer servers, which can enable distributed computing, such as cloud computing. The network 1530, in some cases with the aid of the computer system 1501, can implement a peer-to-peer network, which may enable devices coupled to the computer system 1501 to behave as a client or a server.

The CPU 1505 can execute a sequence of machine-readable instructions, which can be embodied in a program or software. The instructions may be stored in a memory location, such as the memory 1510. The instructions can be directed to the CPU 1505, which can subsequently program or otherwise configure the CPU 1505 to implement methods of the present disclosure. Examples of operations performed by the CPU 1505 can include fetch, decode, execute, and writeback.

The CPU 1505 can be part of a circuit, such as an integrated circuit. One or more other components of the system 1501 can be included in the circuit. In some cases, the circuit is an application specific integrated circuit (ASIC).

The storage unit 1515 can store files, such as drivers, libraries and saved programs. The storage unit 1515 can store user data, e.g., user preferences and user programs. The computer system 1501 in some cases can include one or more additional data storage units that are external to the computer system 1501, such as located on a remote server that is in communication with the computer system 1501 through an intranet or the Internet.

The computer system 1501 can communicate with one or more remote computer systems through the network 1530. For instance, the computer system 1501 can communicate with a remote computer system of a user (e.g., a mobile device). Examples of remote computer systems include personal computers (e.g., portable PC), slate or tablet PC's (e.g., Apple® iPad, Samsung® Galaxy Tab), telephones, Smart phones (e.g., Apple® iphone, Android-enabled device, Blackberry®), or personal digital assistants. The user can access the computer system 1501 via the network 1530.

Methods as described herein can be implemented by way of machine (e.g., computer processor) executable code stored on an electronic storage location of the computer system 1501, such as, for example, on the memory 1510 or electronic storage unit 1515. The machine executable or machine readable code can be provided in the form of software. During use, the code can be executed by the processor 1505. In some cases, the code can be retrieved from the storage unit 1515 and stored on the memory 1510 for ready access by the processor 1505. In some situations, the electronic storage unit 1515 can be precluded, and machine-executable instructions are stored on memory 1510.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 1501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1501 can include or be in communication with an electronic display 1535 that comprises a user interface (UI) 1540 for providing, for example, a recovery score. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1505. The algorithm can, for example, predict a time to recovery.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-19. (canceled)

20. A system for capturing wearable health data, comprising:
(A) a sleep sensor configured to physically couple to a body of a user, wherein said sleep sensor is further configured to monitor sleep of said user while said sleep sensor is physically coupled to said body of said user;
(B) an activity sensor configured to monitor activity of said user; and
(C) a server comprising one or more processors and one or more memories storing computer-executable instructions that, when executed, cause said one or more processors to perform operations comprising:
(i) obtaining sleep data of said user, wherein said sleep data is collected by said sleep sensor over a first time period, wherein at least part of said first time period is at least two weeks after a surgery,
(ii) processing said sleep data to generate sleep efficiency data corresponding to said user over said first time period,
(iii) obtaining activity data of said user, wherein said activity data is collected by said activity sensor over a second time period that is after said surgery,
(iv) processing said activity data to generate step count data corresponding to said user over said second time period,
(v) predicting, via a machine learning model, a recovery trajectory from said surgery for said user based at least in part on processing said sleep efficiency data and said step count data, and
(vi) causing a representation of said recovery trajectory to be displayed to said user via a graphical user interface of a mobile device.

21. The system of claim 20, wherein said sleep sensor is configured to physically couple to a limb of said body of said user.

22. The system of claim 20, wherein said activity sensor comprises one or more of: an orientation sensor, a gyroscope, or an accelerometer.

23. The system of claim 20, wherein said first time period ends at a first time and said second time period ends at a second time, wherein said first time is after said second time.

24. The system of claim 20, wherein said machine learning model predicts said recovery trajectory based at least in part on a ranking, wherein said ranking is determined by operations comprising:
(i) generating a similarity group of a plurality of subjects sharing at least one characteristic with said user, wherein said at least one characteristic relates to health data, personal data, or demographic data; and
(ii) calculating said ranking for said user with respect to said similarity group based at least in part on a comparison of both of said sleep efficiency data of said user and said step count data of said user against both sleep efficiencies of said plurality of subjects and step count data of said plurality of subjects.

25. The system of claim 20, wherein said machine learning model comprises a classifier.

26. The system of claim 25, wherein said classifier comprises one or both of a decision tree or a random forest.

27. The system of claim 25, wherein said classifier was trained using a backpropagation-based gradient update technique that progressively matches output of said classifier with corresponding ground truths of a training set.

28. The system of claim 27, wherein said training set is generated at least in part by:
(i) generating a plurality of vectors for a cohort comprising a plurality of subjects, each vector comprising information associated with at least one characteristic of a subject from said cohort; and
(ii) generating said training set at least in part by processing a subset of vectors of said plurality of vectors with a distance function that determines a similarity of said at least one characteristic between each subject of a subset of subjects of said plurality of subjects.

29. The system of claim 20, further comprising: (E) a cardiovascular sensor configured to monitor cardiovascular activity of said user.

30. The system of claim 29, wherein said computer-executable instructions, when executed, cause said one or more processors to perform further operations comprising:
(i) obtaining cardiovascular data of said user, wherein said cardiovascular data is collected by said cardiovascular sensor over a third time period that is after said surgery, and
(ii) processing said cardiovascular data to generate heart rate data corresponding to said user over said third time period.

31. The system of claim 30, wherein predicting said recovery trajectory from said surgery for said user is further based at least in part on processing said heart rate data.

32. The system of claim 20, wherein processing said sleep data further generates sleep quality data corresponding to said user over said first time period, and wherein said machine learning model predicts said recovery trajectory from said surgery further based at least in part on said sleep quality data.

33. The system of claim 20, wherein said surgery corresponds to one or more of: bone fracture repair, tendon or ligament repair, or joint replacement.

34. The system of claim 20, wherein said sleep sensor is physically separate from said mobile device, and wherein said activity sensor is internal to said mobile device.

35. The system of claim 20, wherein said computer-executable instructions, when executed, cause said one or more processors to perform a health intervention.

36. The system of claim 35, wherein performing said health intervention comprises one or more of: altering a therapeutic regime, altering an exercise regime, arranging a surgery, or providing a personalized recommendation.

37. A system for capturing wearable health data comprising:
(A) a sleep sensor configured to physically couple to a body of a user, wherein said sleep sensor is further configured to monitor sleep of said user while said sleep sensor is physically coupled to said body of said user;
(B) an activity sensor configured to monitor activity of said user; and
(C) A mobile device comprising one or more processors and one or more memories storing computer-executable instructions that, when executed, cause said one or more processors to perform operations comprising:
(a) receiving a recovery trajectory of said user from a surgery, wherein said recovery trajectory is generated using operations comprising:
(i) obtaining sleep data of said user, wherein said sleep data is collected by said sleep sensor over a first time period, wherein at least part of said first time period is at least two weeks after said surgery,
(ii) processing said sleep data to generate sleep efficiency data corresponding to said user over said first time period,
(iii) obtaining activity data of said user, wherein said activity data is collected by said activity sensor over a second time period that is after said surgery,
(iv) processing said activity data to generate step count data corresponding to said user over said second time period, and
(v) predicting, via a machine learning model, said recovery trajectory from said surgery for said user based at least in part on processing said sleep efficiency data and said step count data; and
(b) display a representation of said recovery trajectory to said user via a graphical user interface of said mobile device.

38. The system of claim 37, wherein said computer-executable instructions, when executed, cause said one or more processors to display a notification configured to change a behavior of said user via said graphical user interface.

39. The system of claim 37, further comprising (D) a cardiovascular sensor configured to monitor cardiovascular activity of said user, wherein said recovery trajectory is generated at least in part by obtaining cardiovascular data of said user, wherein said cardiovascular data is collected by said cardiovascular sensor over a third time period that is after said surgery, processing said cardiovascular data to generate heart rate data corresponding to said user over said third time period, and processing said heart rate data.
